(19) United States
(12) Patent Application Publication
(10) Pub. No.: US 2025/0281502 A1
(43) Pub. Date: Sep. 11, 2025

(54) NEW DRUG APPLICATION

(71) Applicant: Centre National de la Recherche Scientifique, PARIS (FR)

(72) Inventors: Laure DUTRIEUX, Montpellier (FR); Jérôme MOREAUX, Montpellier (FR); Philippe PASERO, Montpellier (FR); Raphaël RODRIGUEZ, Paris (FR); Michel COGNÉ, Rennes (FR)

(21) Appl. No.: 18/575,733

(22) PCT Filed: Jul. 1, 2022
(86) PCT No.: PCT/EP2022/068304

§ 371 Date: Dec. 29, 2023

(30) Foreign Application Priority Data
Jul. 1, 2021 (EP) 21305916.5
Jul. 27, 2021 (EP) 21306047.8
Jul. 27, 2021 (FR) FR2108147

(51) Int. Cl. A61K 31/5517; A61K 31/4045; A61K 31/4709; A61P 35/00

(52) U.S. Cl. CPC A61K 31/5517; A61K 31/4045; A61K 31/4709; A61P 35/00

(57) ABSTRACT

A method is disclosed for treating an individual afflicted by a multiple myeloma by a composition comprising at least one G-quadruplex (G4) stabilizer. Also disclosed is a composition comprising at least one G-quadruplex (G4) stabilizer for its use in a method for treating an individual afflicted by a multiple myeloma.

### CROSS-REFERENCE TO RELATED APPLICATIONS

The present application is a filing under 35 U.S.C. 371 as the National Stage of International Application No. PCT/EP2022/068304, filed Jul. 1, 2022, entitled “NEW DRUG APPLICATION”, which claims priority to European Application No. EP 21305916.5 filed with the European Patent Office on Jul. 1, 2021, and entitled “NEW DRUG APPLICATION”; European Application No. EP 21306047.8 filed with the European Patent Office on Jul. 27, 2021, and entitled “NEW DRUG APPLICATION”; and French Application No. FR 2108147 filed with the French Patent Office on Jul. 27, 2021, and entitled “NEW DRUG APPLICATION”, all of which are incorporated herein by reference in their entirety for all purposes.

### INCORPORATION BY REFERENCE OF MATERIAL IN XML FILE

This application incorporates by reference the Sequence Listing contained in the following XML file submitted Dec. 29, 2023:

File name: 4692-15000 Sequence listing.xml; filed on Dec. 29, 2023; and having a file size of 30.7 KB.

The information in the Sequence Listing is incorporated herein in its entirety for all purposes.

The invention relates to a new drug application.

Multiple myeloma (MM) is the second most frequent hematological malignancy, characterized by the accumulation of malignant plasma cells (PCs) within the bone marrow. More than 5000 new cases are identified in France each year, and multiple myeloma represents 2% of cancer-related cell death.

To date, there is no definitive treatment for this pathology and a majority of patients will invariably relapse. Oncogenic transformation in MM is thought to occur within the secondary lymphoid organs. Malignant PCs present a high rate of somatic mutations, suggesting that the oncogenetic event occurs after the end of the somatic hyper-mutation (SHM) process, which physiologically takes place in the germinal centers (GCs). However, the precise molecular events leading to myelomagenesis remain obscure. Broad genomic instability is frequent in MM and not restricted to the IgH locus, but IgH S regions are exquisitely involved in various translocations (notably with MAF, MAFB, CCND1, CCND3, WWOX, FGFR3/MMSET . . . ) which thus stand as late “post-GC” events. These events can yield fused transcripts, the frequency of which is clearly correlated with a bad prognosis. A better understanding of early myelomagenesis and myeloma progression will help define therapeutic areas of interest.

The invention intends to obviate this lack in the art.

The object of the invention is to provide a new efficient drug for treating multiple myeloma.

Thus, the invention relates to a composition comprising at least one G-quadruplex (G4) stabilizer for its use in a method for treating an individual afflicted by a multiple myeloma.

In the invention, the term “individual” refers to a mammal individual, preferably a human individual.

The inventors unexpectedly discovered that a patient with MM can be treated by a therapeutic composition targeting the transcription/replication conflicts (TRCs) resolution cell machinery, and more particularly by stabilising the G4 structures.

TRCs occur at R-loop structures during the replication stage of a cell. R-loops are three-stranded nucleic acid structures, formed by the annealing of an RNA moiety with double-stranded DNA constituting an RNA:DNA hybrid. These structures are physiologically enriched near promoters and transcription termination sites, and are involved in immunoglobulin (Ig) class switch recombination (CSR), transcription initiation and termination, and telomere elongation. Unscheduled R-loop formation interferes with replication fork progression and increases the collision rate between the replication and transcription machineries, known as transcription/replication conflicts (TRCs).

G-quadruplex (G4) are four-stranded secondary DNA structures, constituted of at least two stacked guanine tetrads stabilized by Hoogsteen hydrogen bonds and cations, forming a planar complex (G-quartet). These G-quartets are stabilized by a central counterion, typically K⁺, and stack upon each other forming stable structures. These highly stable non-canonical structures are present at telomeres, at the promoter of many genes, and at replication origins. G4s can be formed in the displaced DNA strand of a R-loop in order to stabilize it.

G4 stabilizers are compounds that avoid the G4 structures to untie, with the result that G4 stabilizers impede with the resolution of R-loop structures and trigger the occurrence of TRCs.

Unexpectedly, the inventors identified that G4 stabilizers are able to induce apoptosis or to inhibit cell cycle of primary cells from individuals afflicted by a multiple myeloma.

In particular, the at least one G4 stabilizer is selected in a group consisting of Quarfloxin, Pidnarulex, MM41 (4,9-Bis((3-(4-methylpiperazin-1-yl)propyl)amino)-2,7-bis(3-morpholinopropyl)benzo[Imn][3,8]phenanthroline-1,3,6,8(2H,7H)-tetraone), Telomestatin, BMSG-SH-3 (2,7-Bis-[5-(4-methyl-piperazin-1-yl)-pentyl]-4,9-bis-[3-(4-methyl-piperazin-1-yl)-propylamino]-benzo[Imn][3,8]phenanthroline-1,3,6,8-tetraone), BRACO-19 (N,N′-(9-(4-(Dimethylamino)phenylamino)acridine-3,6-diyl)bis(3-(pyrrolidin-1-yl)propanamide) hydrochloride), CM03 ((2,7-bis(3-morpholinopropyl)-4-((2-(pyrrolidin-1-yl)ethyl)amino)benzo[Imn][3,8]phenanthroline-1,3,6,8(2H,7H)-tetraone), PDP, Pyridostatin, carboxyPyridostatin, PhenDC3 (3,3′-[1,10-Phenanthroline-2,9-diylbis(carbonylimino)]bis[1-methylquinolinium]1,1,1-trifluoromethanesulfonate (1:2)), AQ1, TMPyP4 (Meso-Tetra (N-methyl-4-pyridyl) porphine tetra tosylate), RHPS4 (3,11-Difluoro-6,8,13-trimethylquino[4,3,2-kl]acridinium methylsulfate), 360A(2-N,6-N-bis(1-methylquinolin-1-ium-3-yl)pyridine-2,6-dicarboxamide), FG (bis-guanylhydrazone derivative of diimidazo[1,2-a:1,2-c]pyrimidine), 20A, Emetine and in particular combinations thereof. In particular, the G4 stabilizer is Pyridostatin.

PDP corresponds to compound 7 in Emanuela Ruggiero and Sara N Richter, Nucleic Acids Research, Volume 46, Issue 7, 20 Apr. 2018, Pages 3270-3283.

AQ1 is described on FIG. 2 in Eleonora Zorzan et al., Oncotarget, 2016, Vol. 7, No. 16.

20A corresponds to compound 3 in N. M. Smith et al., Organic & Biomolecular Chemistry, 2011, issue 17.

Advantageously, the at least one G4 stabilizer is associated with a pharmaceutical acceptable vehicle.

An “acceptable pharmaceutical vehicle” refers in the invention to any carrier, emulsion or excipient that does not impede with the therapeutic effect of the composition nor harm the health of the individual. It is within the skills of a physician to determine the said acceptable pharmaceutical vehicle.

It also is within the skills of a physician to determine the specific therapeutically effective dosage regimen, as this dosage regimen will be dependent upon a variety of factors including, but not limited to: the severity of the multiple myeloma; the age; the body weight; general health; the sex; the diet; the time course of administration; the route of administration; the duration of the treatment; the drugs that are concomitantly administered in combination with the pharmaceutical composition within the scope of the present invention.

In some embodiments, the dosage regimen of said at least one G4 stabilizer ranges from about 0.0001 mg to about 1,000 mg per adult, per day. Preferably, the individual is administered with an amount of about 0.0001, 0.0005, 0.001, 0.005, 0.01, 0.05, 0.1, 0.5, 1.0, 2.5, 5.0, 7.5, 10.0, 15.0, 20.0, 25.0, 50.0, 75.0, 15 100, 250, 500 and 750 mg of said at least one G4 stabilizer in order to adjust the dosage regimen that is the most suitable to a particular individual in need of the treatment.

A pharmaceutical composition within the scope of the present invention may contain from about 0.01 mg to about 500 mg of said at least one G4 stabilizer, preferably from about 1 mg to about 100 mg of said at least one G4 stabilizer.

In a preferred embodiment, an effective amount of at least one G4 stabilizer is routinely administered to an individual in need thereof, at a dosage regimen from about 0.0002 mg/kg to about 20 mg/kg of body weight per day, in particular from about 0.001 mg/kg to 7 mg/kg of body weight per day.

The optimal amount of said at least one G4 stabilizer to be comprised in a pharmaceutical dosage unit according to the invention may be easily adapted by the one skilled in the art using routine known protocols or methods.

Said composition may be administered by any suitable route, i.e. including, but not limited to, an oral, sublingual, subcutaneous, intraperitoneal, intramuscular, intravenous, intrathecal and rectal administration.

In one embodiment of the invention, the composition further comprises a drug commonly used for treating multiple myeloma, and possibly to which a resistance occurs.

In the invention, “a drug commonly used for treating multiple myeloma” refers to anticancer drugs or compounds.

Resistance to a drug, regarding MM, means that said drug is not able to affect survival and/or proliferation of the cells that constitute MM (induce apoptosis and/or necrosis and inhibit cell proliferation). If a resistance occurs, it means that the malignant cells of the MM were initially sensitive to the drug, but further to the treatment, or during the treatment, mutations may occur in some cells, such that the target of the drug is not any more sensitive to the drug. Therefore, the cells become insensitive to the drug and a resistance appears, i.e. the tumor grows from the resistant cells.

In particular, the composition further comprises at least one histone deacetylase inhibitor.

The inventors unexpectedly discovered that stabilizing G4 structures potentializes the histone deacetylase inhibitors cytotoxicity on multiple myeloma cells. The inventors have identified that the combination between at least one G4 stabilizer and at least one histone deacetylase inhibitor is able to induce apoptosis or to inhibit cell cycle of primary cells from individuals afflicted by a multiple myeloma with a synergetic effect.

In the invention, the term “histone deacetylase inhibitor” or “HDACi” refers to histone deacetylase inhibitor that can be grouped in four classes: hydroxamates (panobinostat (LBH-589), trichostatin-A (TSA), vorinostat (SAHA), belinostat (PXDI01), NVP-LAQ824 and givinostat (ITF2357)), cyclic peptide (romidepsin (depsipeptide)), aliphatic acids (valproic acid (VPA) and sodium phenylbutyrate) and benzamides (MS-275, MGCD0103). HDACi are characterized as class I-specific HDACs inhibitors (MGCD0103, romidepsin and MS-275) or as pan-HDAC inhibitors, denoting activity against both classes I and II HDACis (TSA, panobinostat, vorinostat and belinostat).

In particular, the at least one histone deacetylase inhibitor is selected in a group consisting of Panobinostat, trichostatin-A, vorinostat, belinostat, NVP-LAQ824 (Dacinostat), givinostat, romidepsin, valproic acid, sodium phenylbutyrate, MS-275 (N-(2-aminophenyl)-4-[N-(pyridine-3yl-methoxy-carbonyl) aminomethyl]benzamide), MGCDO103 (Mocetinostat), and in particular combinations thereof. Particularly, the histone deacetylase inhibitor is Panobinostat.

Advantageously, the at least one G4 stabilizer and at least one histone deacetylase inhibitor are used simultaneously, separately, or sequentially.

By a simultaneous use, it is meant in the invention that all the compounds are injected or administered to an individual at the same time. Separately use means that the compounds are provided in a separate formulation but are injected or administered at the same time. Sequentially means that the compounds are delivered to the individual separately over the time.

Advantageously, the at least one G4 stabilizer and at least one histone deacetylase inhibitor are associated with a pharmaceutical acceptable vehicle. The pharmaceutical acceptable vehicle is as defined above.

It is within the skills of a physician to determine the specific therapeutically effective dosage regimen, as this dosage regimen will be dependent upon a variety of factors including, but not limited to: the severity of the multiple myeloma; the age; the body weight; general health; the sex; the diet; the time course of administration; the route of administration; the duration of the treatment; the drugs that are concomitantly administered in combination with the pharmaceutical composition within the scope of the present invention.

In some embodiments, the dosage regimen of each of said at least one G4 stabilizer and at least one histone deacetylase inhibitor ranges from about 0.0001 mg to about 1,000 mg per adult, per day. Preferably, the individual is administered with an amount of about 0.0001, 0.0005, 0.001, 0.005, 0.01, 0.05, 0.1, 0.5, 1.0, 2.5, 5.0, 7.5, 10.0, 15.0, 20.0, 25.0, 50.0, 75.0, 15 100, 250, 500 and 750 mg of each of said at least one G4 stabilizer and at least one histone deacetylase inhibitor in order to adjust the dosage regimen that is the most suitable to a particular individual in need of the treatment.

A pharmaceutical composition within the scope of the present invention may contain from about 0.01 mg to about 500 mg of each of the said at least one G4 stabilizer and at least one histone deacetylase inhibitor, preferably from about 1 mg to about 100 mg of said at least one G4 stabilizer and at least one histone deacetylase inhibitor.

In a preferred embodiment, an effective amount of each of the said at least one G4 stabilizer and at least one histone deacetylase inhibitor is routinely administered to an individual in need thereof, at a dosage regimen from about 0.0002 mg/kg to about 20 mg/kg of body weight per day, in particular from about 0.001 mg/kg to 7 mg/kg of body weight per day.

The optimal amount of the said at least one G4 stabilizer and at least one histone deacetylase inhibitor to be comprised in a pharmaceutical dosage unit according to the invention may be easily adapted by the one skilled in the art using routine known protocols or methods.

Said composition may be administered by any suitable route, i.e. including, but not limited to, an oral, sublingual, subcutaneous, intraperitoneal, intramuscular, intravenous, intrathecal and rectal administration.

In particular, the composition further comprises at least one bromodomain and extraterminal (BET) proteins inhibitor.

The inventors unexpectedly discovered that stabilizing G4 structures potentializes the BET proteins inhibitors cytotoxicity on multiple myeloma cells. The inventors have identified that the combination between at least one G4 stabilizer and at least one histone BET proteins inhibitor/deacetylase inhibitor is able to induce apoptosis or to inhibit cell cycle of primary cells from individuals afflicted by a multiple myeloma with a synergetic effect.

BET proteins inhibitors are a class of drugs that reversibly bind the bromodomains of BET proteins BRD2, BRD3, BRD4, and BRDT, and prevent protein-protein interaction between BET proteins and acetylated histones and transcription factors.

Particularly, the at least one BET proteins inhibitor is selected in the group consisting of RVX-208 (2-[4-(2-hydroxyethoxy)-3,5-dimethylphenyl]-5,7-dimethoxy-4(3H)-quinazolinone), I-BET-762 ((4S)-6-(4-Chlorophenyl)-N-ethyl-8-methoxy-1-methyl-4H-[1,2,4]triazolo[4,3-a][1,4]benzodiazepine-4-acetamide), OTX015 ((6S)-4-(4-Chlorophenyl)-N-(4-hydroxyphenyl)-2,3,9-trimethyl-6H-thieno[3,2-f][1,2,4]triazolo[4,3-a][1,4]diazepine-6-acetamide), CPI-0610 (Pelabresib), GSK525762 (Molibresib), ABBV-075 (Mivebresib), FT-1101 ([(2S)-5-(Cyclobutyloxy)-3,4-dihydro-2-methyl-6-[1-(4-piperidinyl)-1H-pyrazol-4-yl]-1(2H)-quinolinyl]cyclopropylmethanone), INCB057643 (2,2,4-trimethyl-8-(6-methyl-7-oxo-1H-pyrrolo[2,3-c]pyridin-4-yl)-6-methylsulfonyl-1,4-benzoxazin-3-one), ZEN003694, GSK2820151, CC-90010 (Trotabresib), PLX51107 (4-[6-(3,5-dimethyl-1,2-oxazol-4-yl)-1-[(1S)-1-pyridin-2-ylethyl]pyrrolo[3,2-b]pyridin-3-yl]benzoic acid), ABBV-744 (N-ethyl-4-[2-(4-fluoro-2,6-dimethylphenoxy)-5-(2-hydroxypropan-2-yl)phenyl]-6-methyl-7-oxo-1H-pyrrolo[2,3-c]pyridine-2-carboxamide), BAY1238097 ((4S)-7,8-dimethoxy-N,4-dimethyl-1-[4-(4-methylpiperazin-1-yl)phenyl]-4,5-dihydro-2,3-benzodiazepine-3-carboxamide), B1894999 (6-[1-benzyl-6-(4-methylpiperazin-1-yl)benzimidazol-2-yl]-N,3-dimethyl-[1,2,4]triazolo[4,3-a]pyrazin-8-amine), BMS-986158 (2-[3-(3,5-dimethyltriazol-4-yl)-5-[(S)-oxan-4-yl(phenyl)methyl]pyrido[3,2-b]indol-7-yl]propan-2-ol), GS-5829 ([2-cyclopropyl-6-(3,5-dimethyl-1,2-oxazol-4-yl)-1H-benzimidazol-4-yl]-dipyridin-2-ylmethanol), INCB054328, R06870810 (2-[(9S)-7-(4-chlorophenyl)-4,5,13-trimethyl-3-thia-1,8,11,12-tetrazatricyclo[8.3.0.02,6]trideca-2(6),4,7,10,12-pentaen-9-yl]-N-[3-(4-methylpiperazin-1-yl)propyl]acetamide), and in particular combinations thereof.

In particular, the at least one BET proteins inhibitor is selected in the group consisting of RVX-208, I-BET-762, OTX015, CPI-0610, GSK525762, ABBV-075, FT-1101, INCB057643, CC-90010, PLX51107, ABBV-744, BAY1238097, B1894999, BMS-986158, GS-5829, R06870810, and combinations thereof.

Advantageously, the at least one G4 stabilizer and at least one BET proteins inhibitor/histone deacetylase inhibitor are used simultaneously, separately, or sequentially.

Advantageously, the at least one G4 stabilizer and at least one BET proteins inhibitor are associated with a pharmaceutical acceptable vehicle. The pharmaceutical acceptable vehicle is as defined above.

It is within the skills of a physician to determine the specific therapeutically effective dosage regimen, as this dosage regimen will be dependent upon a variety of factors including, but not limited to: the severity of the multiple myeloma; the age; the body weight; general health; the sex; the diet; the time course of administration; the route of administration; the duration of the treatment; the drugs that are concomitantly administered in combination with the pharmaceutical composition within the scope of the present invention.

In some embodiments, the dosage regimen of each of said at least one G4 stabilizer and at least one BET proteins inhibitor ranges from about 0.0001 mg to about 1,000 mg per adult, per day. Preferably, the individual is administered with an amount of about 0.0001, 0.0005, 0.001, 0.005, 0.01, 0.05, 0.1, 0.5, 1.0, 2.5, 5.0, 7.5, 10.0, 15.0, 20.0, 25.0, 50.0, 75.0, 15 100, 250, 500 and 750 mg of each of said at least one G4 stabilizer and at least one BET proteins inhibitor/histone deacetylase inhibitor in order to adjust the dosage regimen that is the most suitable to a particular individual in need of the treatment.

A pharmaceutical composition within the scope of the present invention may contain from about 0.01 mg to about 500 mg of each of the said at least one G4 stabilizer and at least one BET proteins inhibitor/histone deacetylase inhibitor, preferably from about 1 mg to about 100 mg of said at least one G4 stabilizer and at least one BET proteins inhibitor/histone deacetylase inhibitor.

In a preferred embodiment, an effective amount of each of the said at least one G4 stabilizer and at least one BET proteins inhibitor is routinely administered to an individual in need thereof, at a dosage regimen from about 0.0002 mg/kg to about 20 mg/kg of body weight per day, in particular from about 0.001 mg/kg to 7 mg/kg of body weight per day.

The optimal amount of the said at least one G4 stabilizer and at least one BET proteins inhibitor to be comprised in a pharmaceutical dosage unit according to the invention may be easily adapted by the one skilled in the art using routine known protocols or methods.

Said composition may be administered by any suitable route, i.e. including, but not limited to, an oral, sublingual, subcutaneous, intraperitoneal, intramuscular, intravenous, intrathecal and rectal administration.

In particular, the composition further comprises at least one nitrogen mustard.

The inventors unexpectedly discovered that stabilizing G4 structures potentializes the nitrogen mustards cytotoxicity on multiple myeloma cells. The inventors have identified that the combination between at least one G4 stabilizer and at least one nitrogen mustard is able to induce apoptosis or to inhibit cell cycle of primary cells from individuals afflicted by a multiple myeloma with a synergetic effect.

Nitrogen mustards nonspecific DNA alkylating agents. They are cytotoxic organic compounds with the chloroethylamine (Cl(CH₂)₂NR₂) functional group which form cyclic aminium ions (aziridinium rings) by intramolecular displacement of the chloride by the amine nitrogen. This aziridinium group then alkylates DNA once it is attacked by the N-7 nucleophilic centre on the guanine base.

Particularly, the at least one nitrogen mustard is selected in the group consisting of Chlormethine, Chlorambucil, Melphalan, Cyclophosphamide, Ifosfamide, Estramustine, Prednimustine, Bendamustine, Melphalan flufenamide (Melflufen) and combinations thereof. In particular, the nitrogen mustard is Melphalan.

Advantageously, the at least one G4 stabilizer and at least one nitrogen mustard inhibitor are used simultaneously, separately, or sequentially.

Advantageously, the at least one G4 stabilizer and at least one nitrogen mustard are associated with a pharmaceutical acceptable vehicle. The pharmaceutical acceptable vehicle is as defined above.

It is within the skills of a physician to determine the specific therapeutically effective dosage regimen, as this dosage regimen will be dependent upon a variety of factors including, but not limited to: the severity of the multiple myeloma; the age; the body weight; general health; the sex; the diet; the time course of administration; the route of administration; the duration of the treatment; the drugs that are concomitantly administered in combination with the pharmaceutical composition within the scope of the present invention.

In some embodiments, the dosage regimen of each of said at least one G4 stabilizer and at least one nitrogen mustard ranges from about 0.0001 mg to about 1,000 mg per adult, per day.

Preferably, the individual is administered with an amount of about 0.0001, 0.0005, 0.001, 0.005, 0.01, 0.05, 0.1, 0.5, 1.0, 2.5, 5.0, 7.5, 10.0, 15.0, 20.0, 25.0, 50.0, 75.0, 15 100, 250, 500 and 750 mg of each of said at least one G4 stabilizer and at least one nitrogen mustard in order to adjust the dosage regimen that is the most suitable to a particular individual in need of the treatment.

A pharmaceutical composition within the scope of the present invention may contain from about 0.01 mg to about 500 mg of each of the said at least one G4 stabilizer and at least one nitrogen mustard, preferably from about 1 mg to about 100 mg of said at least one G4 stabilizer and at least one nitrogen mustard.

The optimal amount of the said at least one G4 stabilizer and at least one nitrogen mustard to be comprised in a pharmaceutical dosage unit according to the invention may be easily adapted by the one skilled in the art using routine known protocols or methods.

Said composition may be administered by any suitable route, i.e. including, but not limited to, an oral, sublingual, subcutaneous, intraperitoneal, intramuscular, intravenous, intrathecal and rectal administration.

In another embodiment, the individual has been prognosed with a poor outcome.

The term “outcome” refers to the survival, the relapse or the death of the individual. The outcome may relate to disease-free survival (DFS), progression-free survival (PFS), event free survival (EFS) or overall survival (OS).

Illustratively, a “poor outcome” may refer to a disease relapse or death of the individual. The disease relapse of multiple myeloma may be defined as an increase in circulating monoclonal peak, an increase in medullary plasmacytosis and the return of one or more clinical evidence (hypercalcemia, renal failure, anaemia and bone tissue injuries). In particular, a “poor outcome” may refer to an overall survival after 1000 days below 80%, after 1500 days below 60% or even after 2500 days below 40%. Particularly, a “poor outcome” may refer to an event free survival after 1000 days below 50%. A “poor outcome” may also refer to a median overall survival of the individual around 1600 days or 55 months. Oppositely, a “good outcome” may refer to survival of the individual, with or without relapse episode. In particular, a “good outcome” may refer to an overall survival after 1000 days over 80%, after 1500 days over 80% or even after 2500 days over 60%. Particularly, a “good outcome” may refer to an event free survival after 1000 days over 50%.

A “good outcome” may also refer to a median overall survival over 2500 days.

The term “overall survival” refers to the length of time from either the date of diagnosis or the beginning of treatment for a disease, such as cancer, that patients diagnosed with the disease are still alive.

The term “event free survival” refers to the length of time after primary treatment for a cancer during which the patient remains free of certain complications or events that the treatment was intended to prevent or delay. These events may include the return of the cancer or the onset of certain symptoms, such as bone pain from cancer that has spread to the bone.

The term “disease-free survival” refers to the length of time after primary treatment for a cancer during which the patient survives without any signs or symptoms of that cancer.

The term “progression-free survival” refers to the length of time during and after the treatment of a disease, such as cancer, that a patient lives with the disease but it does not get worse.

In particular, the poor outcome of the individual is in vitro determined by carrying out the following steps:
a) measuring in a biological sample from said individual, the expression level of n genes, wherein n is an integer from 3 to 9 and wherein the n genes are selected from a group of 9 genes consisting of the nucleic acid sequences SEQ ID NO:1 to SEQ ID NO:9;
b) calculating a score TRC_{score} according to the following formula

$$T R {C}_{s c o r e} = {\sum }_{i = 1}^{n} β i \times C i$$

wherein βi represents the regression β coefficient reference value of one of the n genes selected at step a), wherein Ci=1 if the expression level of the said gene is higher than an expression level of reference and Ci=−1 if the expression level of the said gene is lower than or equal to ELRi,
c) prognosing that
   said individual with a score TCR_{score} higher than a reference value TRC_{ref} is likely to have a poor outcome, or
   said individual with a score TCR_{score} lower than a reference value TRC_{ref} is likely to have a good outcome.

In particular, the poor outcome of the individual is in vitro determined by carrying out the following steps:
a) measuring in a biological sample from said individual, the expression level of 9 genes consisting of the nucleic acid sequences SEQ ID NO:1 to SEQ ID NO:9;
b) calculating a score TRC_{score} according to the following formula

$$T R {C}_{s c o r e} = {\sum }_{i = 1}^{9} β i \times C i$$

wherein βi represents the regression β coefficient reference value for the gene of nucleic acid sequence SEQ ID NO:i,
wherein Ci=1 if the expression level of the gene of nucleic acid sequence SEQ ID NO:i is higher than an expression level of reference and Ci=−1 if the expression level of the gene of nucleic acid sequence SEQ ID NO:i is lower than or equal to ELRi,
c) prognosing that
   said individual with a score TCR_{score} higher than a reference value TRCᵣₑᵣ is likely to have a poor outcome, or
   said individual with a score TCR_{score} lower than a reference value TRC_{ref} is likely to have a good outcome,
      wherein the reference value TRCᵣₑf is of −0.39535.

### Step a)

The 466 different combinations of n genes from the group of 9 genes of the invention are listed in Table 2 below.

TABLE 2

| Comb. | Genes Symbol | | | | | | | | |
|---|---|---|---|---|---|---|---|---|---|
| 1 | BRIP1 | DDX1 | DDX23 | | | | | | |
| 2 | BRIP1 | DDX1 | EXOSC5 | | | | | | |
| 3 | BRIP1 | DDX1 | FANCD2 | | | | | | |
| 4 | BRIP1 | DDX1 | HNRNPU | | | | | | |
| 5 | BRIP1 | DDX1 | PRMT5 | | | | | | |
| 6 | BRIP1 | DDX1 | SRPK2 | | | | | | |
| 7 | BRIP1 | DDX1 | XRN2 | | | | | | |
| 8 | BRIP1 | DDX23 | EXOSC5 | | | | | | |
| 9 | BRIP1 | DDX23 | FANCD2 | | | | | | |
| 10 | BRIP1 | DDX23 | HNRNPU | | | | | | |
| 11 | BRIP1 | DDX23 | PRMT5 | | | | | | |
| 12 | BRIP1 | DDX23 | SRPK2 | | | | | | |
| 13 | BRIP1 | DDX23 | XRN2 | | | | | | |
| 14 | BRIP1 | EXOSC5 | FANCD2 | | | | | | |
| 15 | BRIP1 | EXOSC5 | HNRNPU | | | | | | |
| 16 | BRIP1 | EXOSC5 | PRMT5 | | | | | | |
| 17 | BRIP1 | EXOSC5 | SRPK2 | | | | | | |
| 18 | BRIP1 | EXOSC5 | XRN2 | | | | | | |
| 19 | BRIP1 | FANCD2 | HNRNPU | | | | | | |
| 20 | BRIP1 | FANCD2 | PRMT5 | | | | | | |
| 21 | BRIP1 | FANCD2 | SRPK2 | | | | | | |
| 22 | BRIP1 | FANCD2 | XRN2 | | | | | | |
| 23 | BRIP1 | HNRNPU | PRMT5 | | | | | | |
| 24 | BRIP1 | HNRNPU | SRPK2 | | | | | | |
| 25 | BRIP1 | HNRNPU | XRN2 | | | | | | |
| 26 | BRIP1 | PRMT5 | SRPK2 | | | | | | |
| 27 | BRIP1 | PRMT5 | XRN2 | | | | | | |
| 28 | BRIP1 | SRPK2 | XRN2 | | | | | | |
| 29 | DDX1 | DDX23 | EXOSC5 | | | | | | |
| 30 | DDX1 | DDX23 | FANCD2 | | | | | | |
| 31 | DDX1 | DDX23 | HNRNPU | | | | | | |
| 32 | DDX1 | DDX23 | PRMT5 | | | | | | |
| 33 | DDX1 | DDX23 | SRPK2 | | | | | | |
| 34 | DDX1 | DDX23 | XRN2 | | | | | | |
| 35 | DDX1 | EXOSC5 | FANCD2 | | | | | | |
| 36 | DDX1 | EXOSC5 | HNRNPU | | | | | | |
| 37 | DDX1 | EXOSC5 | PRMT5 | | | | | | |
| 38 | DDX1 | EXOSC5 | SRPK2 | | | | | | |
| 39 | DDX1 | EXOSC5 | XRN2 | | | | | | |
| 40 | DDX1 | FANCD2 | HNRNPU | | | | | | |
| 41 | DDX1 | FANCD2 | PRMT5 | | | | | | |
| 42 | DDX1 | FANCD2 | SRPK2 | | | | | | |
| 43 | DDX1 | FANCD2 | XRN2 | | | | | | |
| 44 | DDX1 | HNRNPU | PRMT5 | | | | | | |
| 45 | DDX1 | HNRNPU | SRPK2 | | | | | | |
| 46 | DDX1 | HNRNPU | XRN2 | | | | | | |
| 47 | DDX1 | PRMT5 | SRPK2 | | | | | | |
| 48 | DDX1 | PRMT5 | XRN2 | | | | | | |
| 49 | DDX1 | SRPK2 | XRN2 | | | | | | |
| 50 | DDX23 | EXOSC5 | FANCD2 | | | | | | |
| 51 | DDX23 | EXOSC5 | HNRNPU | | | | | | |
| 52 | DDX23 | EXOSC5 | PRMT5 | | | | | | |
| 53 | DDX23 | EXOSC5 | SRPK2 | | | | | | |
| 54 | DDX23 | EXOSC5 | XRN2 | | | | | | |
| 55 | DDX23 | FANCD2 | HNRNPU | | | | | | |
| 56 | DDX23 | FANCD2 | PRMT5 | | | | | | |
| 57 | DDX23 | FANCD2 | SRPK2 | | | | | | |
| 58 | DDX23 | FANCD2 | XRN2 | | | | | | |
| 59 | DDX23 | HNRNPU | PRMT5 | | | | | | |
| 60 | DDX23 | HNRNPU | SRPK2 | | | | | | |
| 61 | DDX23 | HNRNPU | XRN2 | | | | | | |
| 62 | DDX23 | PRMT5 | SRPK2 | | | | | | |
| 63 | DDX23 | PRMT5 | XRN2 | | | | | | |
| 64 | DDX23 | SRPK2 | XRN2 | | | | | | |
| 65 | EXOSC5 | FANCD2 | HNRNPU | | | | | | |
| 66 | EXOSC5 | FANCD2 | PRMT5 | | | | | | |
| 67 | EXOSC5 | FANCD2 | SRPK2 | | | | | | |
| 68 | EXOSC5 | FANCD2 | XRN2 | | | | | | |
| 69 | EXOSC5 | HNRNPU | PRMT5 | | | | | | |
| 70 | EXOSC5 | HNRNPU | SRPK2 | | | | | | |
| 71 | EXOSC5 | HNRNPU | XRN2 | | | | | | |
| 72 | EXOSC5 | PRMT5 | SRPK2 | | | | | | |
| 73 | EXOSC5 | PRMT5 | XRN2 | | | | | | |
| 74 | EXOSC5 | SRPK2 | XRN2 | | | | | | |
| 75 | FANCD2 | HNRNPU | PRMT5 | | | | | | |
| 76 | FANCD2 | HNRNPU | SRPK2 | | | | | | |
| 77 | FANCD2 | HNRNPU | XRN2 | | | | | | |
| 78 | FANCD2 | PRMT5 | SRPK2 | | | | | | |
| 79 | FANCD2 | PRMT5 | XRN2 | | | | | | |
| 80 | FANCD2 | SRPK2 | XRN2 | | | | | | |
| 81 | HNRNPU | PRMT5 | SRPK2 | | | | | | |
| 82 | HNRNPU | PRMT5 | XRN2 | | | | | | |
| 83 | HNRNPU | SRPK2 | XRN2 | | | | | | |
| 84 | PRMT5 | SRPK2 | XRN2 | | | | | | |
| 85 | BRIP1 | DDX1 | DDX23 | EXOSC5 | | | | | |
| 86 | BRIP1 | DDX1 | DDX23 | FANCD2 | | | | | |
| 87 | BRIP1 | DDX1 | DDX23 | HNRNPU | | | | | |
| 88 | BRIP1 | DDX1 | DDX23 | PRMT5 | | | | | |
| 89 | BRIP1 | DDX1 | DDX23 | SRPK2 | | | | | |
| 90 | BRIP1 | DDX1 | DDX23 | XRN2 | | | | | |
| 91 | BRIP1 | DDX1 | EXOSC5 | FANCD2 | | | | | |
| 92 | BRIP1 | DDX1 | EXOSC5 | HNRNPU | | | | | |
| 93 | BRIP1 | DDX1 | EXOSC5 | PRMT5 | | | | | |
| 94 | BRIP1 | DDX1 | EXOSC5 | SRPK2 | | | | | |
| 95 | BRIP1 | DDX1 | EXOSC5 | XRN2 | | | | | |
| 96 | BRIP1 | DDX1 | FANCD2 | HNRNPU | | | | | |
| 97 | BRIP1 | DDX1 | FANCD2 | PRMT5 | | | | | |
| 98 | BRIP1 | DDX1 | FANCD2 | SRPK2 | | | | | |
| 99 | BRIP1 | DDX1 | FANCD2 | XRN2 | | | | | |
| 100 | BRIP1 | DDX1 | HNRNPU | PRMT5 | | | | | |
| 101 | BRIP1 | DDX1 | HNRNPU | SRPK2 | | | | | |
| 102 | BRIP1 | DDX1 | HNRNPU | XRN2 | | | | | |
| 103 | BRIP1 | DDX1 | PRMT5 | SRPK2 | | | | | |
| 104 | BRIP1 | DDX1 | PRMT5 | XRN2 | | | | | |
| 105 | BRIP1 | DDX1 | SRPK2 | XRN2 | | | | | |
| 106 | BRIP1 | DDX23 | EXOSC5 | FANCD2 | | | | | |
| 107 | BRIP1 | DDX23 | EXOSC5 | HNRNPU | | | | | |
| 108 | BRIP1 | DDX23 | EXOSC5 | PRMT5 | | | | | |
| 109 | BRIP1 | DDX23 | EXOSC5 | SRPK2 | | | | | |
| 110 | BRIP1 | DDX23 | EXOSC5 | XRN2 | | | | | |
| 111 | BRIP1 | DDX23 | FANCD2 | HNRNPU | | | | | |
| 112 | BRIP1 | DDX23 | FANCD2 | PRMT5 | | | | | |
| 113 | BRIP1 | DDX23 | FANCD2 | SRPK2 | | | | | |
| 114 | BRIP1 | DDX23 | FANCD2 | XRN2 | | | | | |
| 115 | BRIP1 | DDX23 | HNRNPU | PRMT5 | | | | | |
| 116 | BRIP1 | DDX23 | HNRNPU | SRPK2 | | | | | |
| 117 | BRIP1 | DDX23 | HNRNPU | XRN2 | | | | | |
| 118 | BRIP1 | DDX23 | PRMT5 | SRPK2 | | | | | |
| 119 | BRIP1 | DDX23 | PRMT5 | XRN2 | | | | | |
| 120 | BRIP1 | DDX23 | SRPK2 | XRN2 | | | | | |
| 121 | BRIP1 | EXOSC5 | FANCD2 | HNRNPU | | | | | |
| 122 | BRIP1 | EXOSC5 | FANCD2 | PRMT5 | | | | | |
| 123 | BRIP1 | EXOSC5 | FANCD2 | SRPK2 | | | | | |
| 124 | BRIP1 | EXOSC5 | FANCD2 | XRN2 | | | | | |
| 125 | BRIP1 | EXOSC5 | HNRNPU | PRMT5 | | | | | |
| 126 | BRIP1 | EXOSC5 | HNRNPU | SRPK2 | | | | | |
| 127 | BRIP1 | EXOSC5 | HNRNPU | XRN2 | | | | | |
| 128 | BRIP1 | EXOSC5 | PRMT5 | SRPK2 | | | | | |
| 129 | BRIP1 | EXOSC5 | PRMT5 | XRN2 | | | | | |
| 130 | BRIP1 | EXOSC5 | SRPK2 | XRN2 | | | | | |
| 131 | BRIP1 | FANCD2 | HNRNPU | PRMT5 | | | | | |
| 132 | BRIP1 | FANCD2 | HNRNPU | SRPK2 | | | | | |
| 133 | BRIP1 | FANCD2 | HNRNPU | XRN2 | | | | | |
| 134 | BRIP1 | FANCD2 | PRMT5 | SRPK2 | | | | | |
| 135 | BRIP1 | FANCD2 | PRMT5 | XRN2 | | | | | |
| 136 | BRIP1 | FANCD2 | SRPK2 | XRN2 | | | | | |
| 137 | BRIP1 | HNRNPU | PRMT5 | SRPK2 | | | | | |
| 138 | BRIP1 | HNRNPU | PRMT5 | XRN2 | | | | | |
| 139 | BRIP1 | HNRNPU | SRPK2 | XRN2 | | | | | |
| 140 | BRIP1 | PRMT5 | SRPK2 | XRN2 | | | | | |
| 141 | DDX1 | DDX23 | EXOSC5 | FANCD2 | | | | | |
| 142 | DDX1 | DDX23 | EXOSC5 | HNRNPU | | | | | |
| 143 | DDX1 | DDX23 | EXOSC5 | PRMT5 | | | | | |
| 144 | DDX1 | DDX23 | EXOSC5 | SRPK2 | | | | | |
| 145 | DDX1 | DDX23 | EXOSC5 | XRN2 | | | | | |
| 146 | DDX1 | DDX23 | FANCD2 | HNRNPU | | | | | |
| 147 | DDX1 | DDX23 | FANCD2 | PRMT5 | | | | | |
| 148 | DDX1 | DDX23 | FANCD2 | SRPK2 | | | | | |
| 149 | DDX1 | DDX23 | FANCD2 | XRN2 | | | | | |
| 150 | DDX1 | DDX23 | HNRNPU | PRMT5 | | | | | |
| 151 | DDX1 | DDX23 | HNRNPU | SRPK2 | | | | | |
| 152 | DDX1 | DDX23 | HNRNPU | XRN2 | | | | | |
| 153 | DDX1 | DDX23 | PRMT5 | SRPK2 | | | | | |
| 154 | DDX1 | DDX23 | PRMT5 | XRN2 | | | | | |
| 155 | DDX1 | DDX23 | SRPK2 | XRN2 | | | | | |
| 156 | DDX1 | EXOSC5 | FANCD2 | HNRNPU | | | | | |
| 157 | DDX1 | EXOSC5 | FANCD2 | PRMT5 | | | | | |
| 158 | DDX1 | EXOSC5 | FANCD2 | SRPK2 | | | | | |
| 159 | DDX1 | EXOSC5 | FANCD2 | XRN2 | | | | | |
| 160 | DDX1 | EXOSC5 | HNRNPU | PRMT5 | | | | | |
| 161 | DDX1 | EXOSC5 | HNRNPU | SRPK2 | | | | | |
| 162 | DDX1 | EXOSC5 | HNRNPU | XRN2 | | | | | |
| 163 | DDX1 | EXOSC5 | PRMT5 | SRPK2 | | | | | |
| 164 | DDX1 | EXOSC5 | PRMT5 | XRN2 | | | | | |
| 165 | DDX1 | EXOSC5 | SRPK2 | XRN2 | | | | | |
| 166 | DDX1 | FANCD2 | HNRNPU | PRMT5 | | | | | |
| 167 | DDX1 | FANCD2 | HNRNPU | SRPK2 | | | | | |
| 168 | DDX1 | FANCD2 | HNRNPU | XRN2 | | | | | |
| 169 | DDX1 | FANCD2 | PRMT5 | SRPK2 | | | | | |
| 170 | DDX1 | FANCD2 | PRMT5 | XRN2 | | | | | |
| 171 | DDX1 | FANCD2 | SRPK2 | XRN2 | | | | | |
| 172 | DDX1 | HNRNPU | PRMT5 | SRPK2 | | | | | |
| 173 | DDX1 | HNRNPU | PRMT5 | XRN2 | | | | | |
| 174 | DDX1 | HNRNPU | SRPK2 | XRN2 | | | | | |
| 175 | DDX1 | PRMT5 | SRPK2 | XRN2 | | | | | |
| 176 | DDX23 | EXOSC5 | FANCD2 | HNRNPU | | | | | |
| 177 | DDX23 | EXOSC5 | FANCD2 | PRMT5 | | | | | |
| 178 | DDX23 | EXOSC5 | FANCD2 | SRPK2 | | | | | |
| 179 | DDX23 | EXOSC5 | FANCD2 | XRN2 | | | | | |
| 180 | DDX23 | EXOSC5 | HNRNPU | PRMT5 | | | | | |
| 181 | DDX23 | EXOSC5 | HNRNPU | SRPK2 | | | | | |
| 182 | DDX23 | EXOSC5 | HNRNPU | XRN2 | | | | | |
| 183 | DDX23 | EXOSC5 | PRMT5 | SRPK2 | | | | | |
| 184 | DDX23 | EXOSC5 | PRMT5 | XRN2 | | | | | |
| 185 | DDX23 | EXOSC5 | SRPK2 | XRN2 | | | | | |
| 186 | DDX23 | FANCD2 | HNRNPU | PRMT5 | | | | | |
| 187 | DDX23 | FANCD2 | HNRNPU | SRPK2 | | | | | |
| 188 | DDX23 | FANCD2 | HNRNPU | XRN2 | | | | | |
| 189 | DDX23 | FANCD2 | PRMT5 | SRPK2 | | | | | |
| 190 | DDX23 | FANCD2 | PRMT5 | XRN2 | | | | | |
| 191 | DDX23 | FANCD2 | SRPK2 | XRN2 | | | | | |
| 192 | DDX23 | HNRNPU | PRMT5 | SRPK2 | | | | | |
| 193 | DDX23 | HNRNPU | PRMT5 | XRN2 | | | | | |
| 194 | DDX23 | HNRNPU | SRPK2 | XRN2 | | | | | |
| 195 | DDX23 | PRMT5 | SRPK2 | XRN2 | | | | | |
| 196 | EXOSC5 | FANCD2 | HNRNPU | PRMT5 | | | | | |
| 197 | EXOSC5 | FANCD2 | HNRNPU | SRPK2 | | | | | |
| 198 | EXOSC5 | FANCD2 | HNRNPU | XRN2 | | | | | |
| 199 | EXOSC5 | FANCD2 | PRMT5 | SRPK2 | | | | | |
| 200 | EXOSC5 | FANCD2 | PRMT5 | XRN2 | | | | | |
| 201 | EXOSC5 | FANCD2 | SRPK2 | XRN2 | | | | | |
| 202 | EXOSC5 | HNRNPU | PRMT5 | SRPK2 | | | | | |
| 203 | EXOSC5 | HNRNPU | PRMT5 | XRN2 | | | | | |
| 204 | EXOSC5 | HNRNPU | SRPK2 | XRN2 | | | | | |
| 205 | EXOSC5 | PRMT5 | SRPK2 | XRN2 | | | | | |
| 206 | FANCD2 | HNRNPU | PRMT5 | SRPK2 | | | | | |
| 207 | FANCD2 | HNRNPU | PRMT5 | XRN2 | | | | | |
| 208 | FANCD2 | HNRNPU | SRPK2 | XRN2 | | | | | |
| 209 | FANCD2 | PRMT5 | SRPK2 | XRN2 | | | | | |
| 210 | HNRNPU | PRMT5 | SRPK2 | XRN2 | | | | | |
| 211 | BRIP1 | DDX1 | DDX23 | EXOSC5 | FANCD2 | | | | |
| 212 | BRIP1 | DDX1 | DDX23 | EXOSC5 | HNRNPU | | | | |
| 213 | BRIP1 | DDX1 | DDX23 | EXOSC5 | PRMT5 | | | | |
| 214 | BRIP1 | DDX1 | DDX23 | EXOSC5 | SRPK2 | | | | |
| 215 | BRIP1 | DDX1 | DDX23 | EXOSC5 | XRN2 | | | | |
| 216 | BRIP1 | DDX1 | DDX23 | FANCD2 | HNRNPU | | | | |
| 217 | BRIP1 | DDX1 | DDX23 | FANCD2 | PRMT5 | | | | |
| 218 | BRIP1 | DDX1 | DDX23 | FANCD2 | SRPK2 | | | | |
| 219 | BRIP1 | DDX1 | DDX23 | FANCD2 | XRN2 | | | | |
| 220 | BRIP1 | DDX1 | DDX23 | HNRNPU | PRMT5 | | | | |
| 221 | BRIP1 | DDX1 | DDX23 | HNRNPU | SRPK2 | | | | |
| 222 | BRIP1 | DDX1 | DDX23 | HNRNPU | XRN2 | | | | |
| 223 | BRIP1 | DDX1 | DDX23 | PRMT5 | SRPK2 | | | | |
| 224 | BRIP1 | DDX1 | DDX23 | PRMT5 | XRN2 | | | | |
| 225 | BRIP1 | DDX1 | DDX23 | SRPK2 | XRN2 | | | | |
| 226 | BRIP1 | DDX1 | EXOSC5 | FANCD2 | HNRNPU | | | | |
| 227 | BRIP1 | DDX1 | EXOSC5 | FANCD2 | PRMT5 | | | | |
| 228 | BRIP1 | DDX1 | EXOSC5 | FANCD2 | SRPK2 | | | | |
| 229 | BRIP1 | DDX1 | EXOSC5 | FANCD2 | XRN2 | | | | |
| 230 | BRIP1 | DDX1 | EXOSC5 | HNRNPU | PRMT5 | | | | |
| 231 | BRIP1 | DDX1 | EXOSC5 | HNRNPU | SRPK2 | | | | |
| 232 | BRIP1 | DDX1 | EXOSC5 | HNRNPU | XRN2 | | | | |
| 233 | BRIP1 | DDX1 | EXOSC5 | PRMT5 | SRPK2 | | | | |
| 234 | BRIP1 | DDX1 | EXOSC5 | PRMT5 | XRN2 | | | | |
| 235 | BRIP1 | DDX1 | EXOSC5 | SRPK2 | XRN2 | | | | |
| 236 | BRIP1 | DDX1 | FANCD2 | HNRNPU | PRMT5 | | | | |
| 237 | BRIP1 | DDX1 | FANCD2 | HNRNPU | SRPK2 | | | | |
| 238 | BRIP1 | DDX1 | FANCD2 | HNRNPU | XRN2 | | | | |
| 239 | BRIP1 | DDX1 | FANCD2 | PRMT5 | SRPK2 | | | | |
| 240 | BRIP1 | DDX1 | FANCD2 | PRMT5 | XRN2 | | | | |
| 241 | BRIP1 | DDX1 | FANCD2 | SRPK2 | XRN2 | | | | |
| 242 | BRIP1 | DDX1 | HNRNPU | PRMT5 | SRPK2 | | | | |
| 243 | BRIP1 | DDX1 | HNRNPU | PRMT5 | XRN2 | | | | |
| 244 | BRIP1 | DDX1 | HNRNPU | SRPK2 | XRN2 | | | | |
| 245 | BRIP1 | DDX1 | PRMT5 | SRPK2 | XRN2 | | | | |
| 246 | BRIP1 | DDX23 | EXOSC5 | FANCD2 | HNRNPU | | | | |
| 247 | BRIP1 | DDX23 | EXOSC5 | FANCD2 | PRMT5 | | | | |
| 248 | BRIP1 | DDX23 | EXOSC5 | FANCD2 | SRPK2 | | | | |
| 249 | BRIP1 | DDX23 | EXOSC5 | FANCD2 | XRN2 | | | | |
| 250 | BRIP1 | DDX23 | EXOSC5 | HNRNPU | PRMT5 | | | | |
| 251 | BRIP1 | DDX23 | EXOSC5 | HNRNPU | SRPK2 | | | | |
| 252 | BRIP1 | DDX23 | EXOSC5 | HNRNPU | XRN2 | | | | |
| 253 | BRIP1 | DDX23 | EXOSC5 | PRMT5 | SRPK2 | | | | |
| 254 | BRIP1 | DDX23 | EXOSC5 | PRMT5 | XRN2 | | | | |
| 255 | BRIP1 | DDX23 | EXOSC5 | SRPK2 | XRN2 | | | | |
| 256 | BRIP1 | DDX23 | FANCD2 | HNRNPU | PRMT5 | | | | |
| 257 | BRIP1 | DDX23 | FANCD2 | HNRNPU | SRPK2 | | | | |
| 258 | BRIP1 | DDX23 | FANCD2 | HNRNPU | XRN2 | | | | |
| 259 | BRIP1 | DDX23 | FANCD2 | PRMT5 | SRPK2 | | | | |
| 260 | BRIP1 | DDX23 | FANCD2 | PRMT5 | XRN2 | | | | |
| 261 | BRIP1 | DDX23 | FANCD2 | SRPK2 | XRN2 | | | | |
| 262 | BRIP1 | DDX23 | HNRNPU | PRMT5 | SRPK2 | | | | |
| 263 | BRIP1 | DDX23 | HNRNPU | PRMT5 | XRN2 | | | | |
| 264 | BRIP1 | DDX23 | HNRNPU | SRPK2 | XRN2 | | | | |
| 265 | BRIP1 | DDX23 | PRMT5 | SRPK2 | XRN2 | | | | |
| 266 | BRIP1 | EXOSC5 | FANCD2 | HNRNPU | PRMT5 | | | | |
| 267 | BRIP1 | EXOSC5 | FANCD2 | HNRNPU | SRPK2 | | | | |
| 268 | BRIP1 | EXOSC5 | FANCD2 | HNRNPU | XRN2 | | | | |
| 269 | BRIP1 | EXOSC5 | FANCD2 | PRMT5 | SRPK2 | | | | |
| 270 | BRIP1 | EXOSC5 | FANCD2 | PRMT5 | XRN2 | | | | |
| 271 | BRIP1 | EXOSC5 | FANCD2 | SRPK2 | XRN2 | | | | |
| 272 | BRIP1 | EXOSC5 | HNRNPU | PRMT5 | SRPK2 | | | | |
| 273 | BRIP1 | EXOSC5 | HNRNPU | PRMT5 | XRN2 | | | | |
| 274 | BRIP1 | EXOSC5 | HNRNPU | SRPK2 | XRN2 | | | | |
| 275 | BRIP1 | EXOSC5 | PRMT5 | SRPK2 | XRN2 | | | | |
| 276 | BRIP1 | FANCD2 | HNRNPU | PRMT5 | SRPK2 | | | | |
| 277 | BRIP1 | FANCD2 | HNRNPU | PRMT5 | XRN2 | | | | |
| 278 | BRIP1 | FANCD2 | HNRNPU | SRPK2 | XRN2 | | | | |
| 279 | BRIP1 | FANCD2 | PRMT5 | SRPK2 | XRN2 | | | | |
| 280 | BRIP1 | HNRNPU | PRMT5 | SRPK2 | XRN2 | | | | |
| 281 | DDX1 | DDX23 | EXOSC5 | FANCD2 | HNRNPU | | | | |
| 282 | DDX1 | DDX23 | EXOSC5 | FANCD2 | PRMT5 | | | | |
| 283 | DDX1 | DDX23 | EXOSC5 | FANCD2 | SRPK2 | | | | |
| 284 | DDX1 | DDX23 | EXOSC5 | FANCD2 | XRN2 | | | | |
| 285 | DDX1 | DDX23 | EXOSC5 | HNRNPU | PRMT5 | | | | |
| 286 | DDX1 | DDX23 | EXOSC5 | HNRNPU | SRPK2 | | | | |
| 287 | DDX1 | DDX23 | EXOSC5 | HNRNPU | XRN2 | | | | |
| 288 | DDX1 | DDX23 | EXOSC5 | PRMT5 | SRPK2 | | | | |
| 289 | DDX1 | DDX23 | EXOSC5 | PRMT5 | XRN2 | | | | |
| 290 | DDX1 | DDX23 | EXOSC5 | SRPK2 | XRN2 | | | | |
| 291 | DDX1 | DDX23 | FANCD2 | HNRNPU | PRMT5 | | | | |
| 292 | DDX1 | DDX23 | FANCD2 | HNRNPU | SRPK2 | | | | |
| 293 | DDX1 | DDX23 | FANCD2 | HNRNPU | XRN2 | | | | |
| 294 | DDX1 | DDX23 | FANCD2 | PRMT5 | SRPK2 | | | | |
| 295 | DDX1 | DDX23 | FANCD2 | PRMT5 | XRN2 | | | | |
| 296 | DDX1 | DDX23 | FANCD2 | SRPK2 | XRN2 | | | | |
| 297 | DDX1 | DDX23 | HNRNPU | PRMT5 | SRPK2 | | | | |
| 298 | DDX1 | DDX23 | HNRNPU | PRMT5 | XRN2 | | | | |
| 299 | DDX1 | DDX23 | HNRNPU | SRPK2 | XRN2 | | | | |
| 300 | DDX1 | DDX23 | PRMT5 | SRPK2 | XRN2 | | | | |
| 301 | DDX1 | EXOSC5 | FANCD2 | HNRNPU | PRMT5 | | | | |
| 302 | DDX1 | EXOSC5 | FANCD2 | HNRNPU | SRPK2 | | | | |
| 303 | DDX1 | EXOSC5 | FANCD2 | HNRNPU | XRN2 | | | | |
| 304 | DDX1 | EXOSC5 | FANCD2 | PRMT5 | SRPK2 | | | | |
| 305 | DDX1 | EXOSC5 | FANCD2 | PRMT5 | XRN2 | | | | |
| 306 | DDX1 | EXOSC5 | FANCD2 | SRPK2 | XRN2 | | | | |
| 307 | DDX1 | EXOSC5 | HNRNPU | PRMT5 | SRPK2 | | | | |
| 308 | DDX1 | EXOSC5 | HNRNPU | PRMT5 | XRN2 | | | | |
| 309 | DDX1 | EXOSC5 | HNRNPU | SRPK2 | XRN2 | | | | |
| 310 | DDX1 | EXOSC5 | PRMT5 | SRPK2 | XRN2 | | | | |
| 311 | DDX1 | FANCD2 | HNRNPU | PRMT5 | SRPK2 | | | | |
| 312 | DDX1 | FANCD2 | HNRNPU | PRMT5 | XRN2 | | | | |
| 313 | DDX1 | FANCD2 | HNRNPU | SRPK2 | XRN2 | | | | |
| 314 | DDX1 | FANCD2 | PRMT5 | SRPK2 | XRN2 | | | | |
| 315 | DDX1 | HNRNPU | PRMT5 | SRPK2 | XRN2 | | | | |
| 316 | DDX23 | EXOSC5 | FANCD2 | HNRNPU | PRMT5 | | | | |
| 317 | DDX23 | EXOSC5 | FANCD2 | HNRNPU | SRPK2 | | | | |
| 318 | DDX23 | EXOSC5 | FANCD2 | HNRNPU | XRN2 | | | | |
| 319 | DDX23 | EXOSC5 | FANCD2 | PRMT5 | SRPK2 | | | | |
| 320 | DDX23 | EXOSC5 | FANCD2 | PRMT5 | XRN2 | | | | |
| 321 | DDX23 | EXOSC5 | FANCD2 | SRPK2 | XRN2 | | | | |
| 322 | DDX23 | EXOSC5 | HNRNPU | PRMT5 | SRPK2 | | | | |
| 323 | DDX23 | EXOSC5 | HNRNPU | PRMT5 | XRN2 | | | | |
| 324 | DDX23 | EXOSC5 | HNRNPU | SRPK2 | XRN2 | | | | |
| 325 | DDX23 | EXOSC5 | PRMT5 | SRPK2 | XRN2 | | | | |
| 326 | DDX23 | FANCD2 | HNRNPU | PRMT5 | SRPK2 | | | | |
| 327 | DDX23 | FANCD2 | HNRNPU | PRMT5 | XRN2 | | | | |
| 328 | DDX23 | FANCD2 | HNRNPU | SRPK2 | XRN2 | | | | |
| 329 | DDX23 | FANCD2 | PRMT5 | SRPK2 | XRN2 | | | | |
| 330 | DDX23 | HNRNPU | PRMT5 | SRPK2 | XRN2 | | | | |
| 331 | EXOSC5 | FANCD2 | HNRNPU | PRMT5 | SRPK2 | | | | |
| 332 | EXOSC5 | FANCD2 | HNRNPU | PRMT5 | XRN2 | | | | |
| 333 | EXOSC5 | FANCD2 | HNRNPU | SRPK2 | XRN2 | | | | |
| 334 | EXOSC5 | FANCD2 | PRMT5 | SRPK2 | XRN2 | | | | |
| 335 | EXOSC5 | HNRNPU | PRMT5 | SRPK2 | XRN2 | | | | |
| 336 | FANCD2 | HNRNPU | PRMT5 | SRPK2 | XRN2 | | | | |
| 337 | BRIP1 | DDX1 | DDX23 | EXOSC5 | FANCD2 | HNRNPU | | | |
| 338 | BRIP1 | DDX1 | DDX23 | EXOSC5 | FANCD2 | PRMT5 | | | |
| 339 | BRIP1 | DDX1 | DDX23 | EXOSC5 | FANCD2 | SRPK2 | | | |
| 340 | BRIP1 | DDX1 | DDX23 | EXOSC5 | FANCD2 | XRN2 | | | |
| 341 | BRIP1 | DDX1 | DDX23 | EXOSC5 | HNRNPU | PRMT5 | | | |
| 342 | BRIP1 | DDX1 | DDX23 | EXOSC5 | HNRNPU | SRPK2 | | | |
| 343 | BRIP1 | DDX1 | DDX23 | EXOSC5 | HNRNPU | XRN2 | | | |
| 344 | BRIP1 | DDX1 | DDX23 | EXOSC5 | PRMT5 | SRPK2 | | | |
| 345 | BRIP1 | DDX1 | DDX23 | EXOSC5 | PRMT5 | XRN2 | | | |
| 346 | BRIP1 | DDX1 | DDX23 | EXOSC5 | SRPK2 | XRN2 | | | |
| 347 | BRIP1 | DDX1 | DDX23 | FANCD2 | HNRNPU | PRMT5 | | | |
| 348 | BRIP1 | DDX1 | DDX23 | FANCD2 | HNRNPU | SRPK2 | | | |
| 349 | BRIP1 | DDX1 | DDX23 | FANCD2 | HNRNPU | XRN2 | | | |
| 350 | BRIP1 | DDX1 | DDX23 | FANCD2 | PRMT5 | SRPK2 | | | |
| 351 | BRIP1 | DDX1 | DDX23 | FANCD2 | PRMT5 | XRN2 | | | |
| 352 | BRIP1 | DDX1 | DDX23 | FANCD2 | SRPK2 | XRN2 | | | |
| 353 | BRIP1 | DDX1 | DDX23 | HNRNPU | PRMT5 | SRPK2 | | | |
| 354 | BRIP1 | DDX1 | DDX23 | HNRNPU | PRMT5 | XRN2 | | | |
| 355 | BRIP1 | DDX1 | DDX23 | HNRNPU | SRPK2 | XRN2 | | | |
| 356 | BRIP1 | DDX1 | DDX23 | PRMT5 | SRPK2 | XRN2 | | | |
| 357 | BRIP1 | DDX1 | EXOSC5 | FANCD2 | HNRNPU | PRMT5 | | | |
| 358 | BRIP1 | DDX1 | EXOSC5 | FANCD2 | HNRNPU | SRPK2 | | | |
| 359 | BRIP1 | DDX1 | EXOSC5 | FANCD2 | HNRNPU | XRN2 | | | |
| 360 | BRIP1 | DDX1 | EXOSC5 | FANCD2 | PRMT5 | SRPK2 | | | |
| 361 | BRIP1 | DDX1 | EXOSC5 | FANCD2 | PRMT5 | XRN2 | | | |
| 362 | BRIP1 | DDX1 | EXOSC5 | FANCD2 | SRPK2 | XRN2 | | | |
| 363 | BRIP1 | DDX1 | EXOSC5 | HNRNPU | PRMT5 | SRPK2 | | | |
| 364 | BRIP1 | DDX1 | EXOSC5 | HNRNPU | PRMT5 | XRN2 | | | |
| 365 | BRIP1 | DDX1 | EXOSC5 | HNRNPU | SRPK2 | XRN2 | | | |
| 366 | BRIP1 | DDX1 | EXOSC5 | PRMT5 | SRPK2 | XRN2 | | | |
| 367 | BRIP1 | DDX1 | FANCD2 | HNRNPU | PRMT5 | SRPK2 | | | |
| 368 | BRIP1 | DDX1 | FANCD2 | HNRNPU | PRMT5 | XRN2 | | | |
| 369 | BRIP1 | DDX1 | FANCD2 | HNRNPU | SRPK2 | XRN2 | | | |
| 370 | BRIP1 | DDX1 | FANCD2 | PRMT5 | SRPK2 | XRN2 | | | |
| 371 | BRIP1 | DDX1 | HNRNPU | PRMT5 | SRPK2 | XRN2 | | | |
| 372 | BRIP1 | DDX23 | EXOSC5 | FANCD2 | HNRNPU | PRMT5 | | | |
| 373 | BRIP1 | DDX23 | EXOSC5 | FANCD2 | HNRNPU | SRPK2 | | | |
| 374 | BRIP1 | DDX23 | EXOSC5 | FANCD2 | HNRNPU | XRN2 | | | |
| 375 | BRIP1 | DDX23 | EXOSC5 | FANCD2 | PRMT5 | SRPK2 | | | |
| 376 | BRIP1 | DDX23 | EXOSC5 | FANCD2 | PRMT5 | XRN2 | | | |
| 377 | BRIP1 | DDX23 | EXOSC5 | FANCD2 | SRPK2 | XRN2 | | | |
| 378 | BRIP1 | DDX23 | EXOSC5 | HNRNPU | PRMT5 | SRPK2 | | | |
| 379 | BRIP1 | DDX23 | EXOSC5 | HNRNPU | PRMT5 | XRN2 | | | |
| 380 | BRIP1 | DDX23 | EXOSC5 | HNRNPU | SRPK2 | XRN2 | | | |
| 381 | BRIP1 | DDX23 | EXOSC5 | PRMT5 | SRPK2 | XRN2 | | | |
| 382 | BRIP1 | DDX23 | FANCD2 | HNRNPU | PRMT5 | SRPK2 | | | |
| 383 | BRIP1 | DDX23 | FANCD2 | HNRNPU | PRMT5 | XRN2 | | | |
| 384 | BRIP1 | DDX23 | FANCD2 | HNRNPU | SRPK2 | XRN2 | | | |
| 385 | BRIP1 | DDX23 | FANCD2 | PRMT5 | SRPK2 | XRN2 | | | |
| 386 | BRIP1 | DDX23 | HNRNPU | PRMT5 | SRPK2 | XRN2 | | | |
| 387 | BRIP1 | EXOSC5 | FANCD2 | HNRNPU | PRMT5 | SRPK2 | | | |
| 388 | BRIP1 | EXOSC5 | FANCD2 | HNRNPU | PRMT5 | XRN2 | | | |
| 389 | BRIP1 | EXOSC5 | FANCD2 | HNRNPU | SRPK2 | XRN2 | | | |
| 390 | BRIP1 | EXOSC5 | FANCD2 | PRMT5 | SRPK2 | XRN2 | | | |
| 391 | BRIP1 | EXOSC5 | HNRNPU | PRMT5 | SRPK2 | XRN2 | | | |
| 392 | BRIP1 | FANCD2 | HNRNPU | PRMT5 | SRPK2 | XRN2 | | | |
| 393 | DDX1 | DDX23 | EXOSC5 | FANCD2 | HNRNPU | PRMT5 | | | |
| 394 | DDX1 | DDX23 | EXOSC5 | FANCD2 | HNRNPU | SRPK2 | | | |
| 395 | DDX1 | DDX23 | EXOSC5 | FANCD2 | HNRNPU | XRN2 | | | |
| 396 | DDX1 | DDX23 | EXOSC5 | FANCD2 | PRMT5 | SRPK2 | | | |
| 397 | DDX1 | DDX23 | EXOSC5 | FANCD2 | PRMT5 | XRN2 | | | |
| 398 | DDX1 | DDX23 | EXOSC5 | FANCD2 | SRPK2 | XRN2 | | | |
| 399 | DDX1 | DDX23 | EXOSC5 | HNRNPU | PRMT5 | SRPK2 | | | |
| 400 | DDX1 | DDX23 | EXOSC5 | HNRNPU | PRMT5 | XRN2 | | | |
| 401 | DDX1 | DDX23 | EXOSC5 | HNRNPU | SRPK2 | XRN2 | | | |
| 402 | DDX1 | DDX23 | EXOSC5 | PRMT5 | SRPK2 | XRN2 | | | |
| 403 | DDX1 | DDX23 | FANCD2 | HNRNPU | PRMT5 | SRPK2 | | | |
| 404 | DDX1 | DDX23 | FANCD2 | HNRNPU | PRMT5 | XRN2 | | | |
| 405 | DDX1 | DDX23 | FANCD2 | HNRNPU | SRPK2 | XRN2 | | | |
| 406 | DDX1 | DDX23 | FANCD2 | PRMT5 | SRPK2 | XRN2 | | | |
| 407 | DDX1 | DDX23 | HNRNPU | PRMT5 | SRPK2 | XRN2 | | | |
| 408 | DDX1 | EXOSC5 | FANCD2 | HNRNPU | PRMT5 | SRPK2 | | | |
| 409 | DDX1 | EXOSC5 | FANCD2 | HNRNPU | PRMT5 | XRN2 | | | |
| 410 | DDX1 | EXOSC5 | FANCD2 | HNRNPU | SRPK2 | XRN2 | | | |
| 411 | DDX1 | EXOSC5 | FANCD2 | PRMT5 | SRPK2 | XRN2 | | | |
| 412 | DDX1 | EXOSC5 | HARNPU | PRMT5 | SRPK2 | XRN2 | | | |
| 413 | DDX1 | FANCD2 | HNRNPU | PRMT5 | SRPK2 | XRN2 | | | |
| 414 | DDX23 | EXOSC5 | FANCD2 | HNRNPU | PRMT5 | SRPK2 | | | |
| 415 | DDX23 | EXOSC5 | FANCD2 | HNRNPU | PRMT5 | XRN2 | | | |
| 416 | DDX23 | EXOSC5 | FANCD2 | HNRNPU | SRPK2 | XRN2 | | | |
| 417 | DDX23 | EXOSC5 | FANCD2 | PRMT5 | SRPK2 | XRN2 | | | |
| 418 | DDX23 | EXOSC5 | HNRNPU | PRMT5 | SRPK2 | XRN2 | | | |
| 419 | DDX23 | FANCD2 | HNRNPU | PRMT5 | SRPK2 | XRN2 | | | |
| 420 | EXOSC5 | FANCD2 | HNRNPU | PRMT5 | SRPK2 | XRN2 | | | |
| 421 | BRIP1 | DDX1 | DDX23 | EXOSC5 | FANCD2 | HNRNPU | PRMT5 | | |
| 422 | BRIP1 | DDX1 | DDX23 | EXOSC5 | FANCD2 | HNRNPU | SRPK2 | | |
| 423 | BRIP1 | DDX1 | DDX23 | EXOSC5 | FANCD2 | HNRNPU | XRN2 | | |
| 424 | BRIP1 | DDX1 | DDX23 | EXOSC5 | FANCD2 | PRMT5 | SRPK2 | | |
| 425 | BRIP1 | DDX1 | DDX23 | EXOSC5 | FANCD2 | PRMT5 | XRN2 | | |
| 426 | BRIP1 | DDX1 | DDX23 | EXOSC5 | FANCD2 | SRPK2 | XRN2 | | |
| 427 | BRIP1 | DDX1 | DDX23 | EXOSC5 | HNRNPU | PRMT5 | SRPK2 | | |
| 428 | BRIP1 | DDX1 | DDX23 | EXOSC5 | HNRNPU | PRMT5 | XRN2 | | |
| 429 | BRIP1 | DDX1 | DDX23 | EXOSC5 | HNRNPU | SRPK2 | XRN2 | | |
| 430 | BRIP1 | DDX1 | DDX23 | EXOSC5 | PRMT5 | SRPK2 | XRN2 | | |
| 431 | BRIP1 | DDX1 | DDX23 | FANCD2 | HNRNPU | PRMT5 | SRPK2 | | |
| 432 | BRIP1 | DDX1 | DDX23 | FANCD2 | HNRNPU | PRMT5 | XRN2 | | |
| 433 | BRIP1 | DDX1 | DDX23 | FANCD2 | HNRNPU | SRPK2 | XRN2 | | |
| 434 | BRIP1 | DDX1 | DDX23 | FANCD2 | PRMT5 | SRPK2 | XRN2 | | |
| 435 | BRIP1 | DDX1 | DDX23 | HNRNPU | PRMT5 | SRPK2 | XRN2 | | |
| 436 | BRIP1 | DDX1 | EXOSC5 | FANCD2 | HNRNPU | PRMT5 | SRPK2 | | |
| 437 | BRIP1 | DDX1 | EXOSC5 | FANCD2 | HNRNPU | PRMT5 | XRN2 | | |
| 438 | BRIP1 | DDX1 | EXOSC5 | FANCD2 | HNRNPU | SRPK2 | XRN2 | | |
| 439 | BRIP1 | DDX1 | EXOSC5 | FANCD2 | PRMT5 | SRPK2 | XRN2 | | |
| 440 | BRIP1 | DDX1 | EXOSC5 | HNRNPU | PRMT5 | SRPK2 | XRN2 | | |
| 441 | BRIP1 | DDX1 | FANCD2 | HNRNPU | PRMT5 | SRPK2 | XRN2 | | |
| 442 | BRIP1 | DDX23 | EXOSC5 | FANCD2 | HNRNPU | PRMT5 | SRPK2 | | |
| 443 | BRIP1 | DDX23 | EXOSC5 | FANCD2 | HNRNPU | PRMT5 | XRN2 | | |
| 444 | BRIP1 | DDX23 | EXOSC5 | FANCD2 | HNRNPU | SRPK2 | XRN2 | | |
| 445 | BRIP1 | DDX23 | EXOSC5 | FANCD2 | PRMT5 | SRPK2 | XRN2 | | |
| 446 | BRIP1 | DDX23 | EXOSC5 | HNRNPU | PRMT5 | SRPK2 | XRN2 | | |
| 447 | BRIP1 | DDX23 | FANCD2 | HNRNPU | PRMT5 | SRPK2 | XRN2 | | |
| 448 | BRIP1 | EXOSC5 | FANCD2 | HNRNPU | PRMT5 | SRPK2 | XRN2 | | |
| 449 | DDX1 | DDX23 | EXOSC5 | FANCD2 | HNRNPU | PRMT5 | SRPK2 | | |
| 450 | DDX1 | DDX23 | EXOSC5 | FANCD2 | HNRNPU | PRMT5 | XRN2 | | |
| 451 | DDX1 | DDX23 | EXOSC5 | FANCD2 | HNRNPU | SRPK2 | XRN2 | | |
| 452 | DDX1 | DDX23 | EXOSC5 | FANCD2 | PRMT5 | SRPK2 | XRN2 | | |
| 453 | DDX1 | DDX23 | EXOSC5 | HNRNPU | PRMT5 | SRPK2 | XRN2 | | |
| 454 | DDX1 | DDX23 | FANCD2 | HNRNPU | PRMT5 | SRPK2 | XRN2 | | |
| 455 | DDX1 | EXOSC5 | FANCD2 | HNRNPU | PRMT5 | SRPK2 | XRN2 | | |
| 456 | DDX23 | EXOSC5 | FANCD2 | HNRNPU | PRMT5 | SRPK2 | XRN2 | | |
| 457 | BRIP1 | DDX1 | DDX23 | EXOSC5 | FANCD2 | HNRNPU | PRMT5 | SRPK2 | |
| 458 | BRIP1 | DDX1 | DDX23 | EXOSC5 | FANCD2 | HNRNPU | PRMT5 | XRN2 | |
| 459 | BRIP1 | DDX1 | DDX23 | EXOSC5 | FANCD2 | HNRNPU | SRPK2 | XRN2 | |
| 460 | BRIP1 | DDX1 | DDX23 | EXOSC5 | FANCD2 | PRMT5 | SRPK2 | XRN2 | |
| 461 | BRIP1 | DDX1 | DDX23 | EXOSC5 | HNRNPU | PRMT5 | SRPK2 | XRN2 | |
| 462 | BRIP1 | DDX1 | DDX23 | FANCD2 | HNRNPU | PRMT5 | SRPK2 | XRN2 | |
| 463 | BRIP1 | DDX1 | EXOSC5 | FANCD2 | HNRNPU | PRMT5 | SRPK2 | XRN2 | |
| 464 | BRIP1 | DDX23 | EXOSC5 | FANCD2 | HNRNPU | PRMT5 | SRPK2 | XRN2 | |
| 465 | DDX1 | DDX23 | EXOSC5 | FANCD2 | HNRNPU | PRMT5 | SRPK2 | XRN2 | |
| 466 | BRIP1 | DDX1 | DDX23 | EXOSC5 | FANCD2 | HNRNPU | PRMT5 | SRPK2 | XRN2 |

In the invention, a “biological sample” refers to a biological sample obtained, reached, collected or isolated from an individual, in vivo or in situ. Such samples may be, but not limited to, organs, tissues, fractions thereof and cells isolated from an individual. For example, suitable biological samples include but are not limited to a cell culture, a cell line, a tissue biopsy such as a bone marrow aspirate, a biological fluid such as a blood, pleural effusion or a serum sample, and the like. An advantageous biological sample includes but is not limited to a blood sample, a tissue biopsy, including a bone marrow aspirate. The biological sample as defined in the invention may be a crude sample, or may be purified to various degrees prior to storage, processing, or measurement.

The expression level of the n genes is measured by well-known protocol in the art. These methods are for instance, DNA-CHIPs containing probesets of said n genes, so that an expression level can be determined for each of said n genes. Other methods can be used, such that quantitative PCR strategy by using specific couples of primers for each of said n genes, with either a specific Taqman probe for each of said 9 genes, or SYBR® compounds.

Advantageously, the expression level can be evaluated by measuring the expression level of mRNA for each of the n genes. This measurement may be carried out by using the well-known techniques available in the art. In this case, mRNA may be extracted, for example using lytic enzymes or chemical solutions or extracted by commercially available nucleic-acid-binding resins following the manufacturer's instructions. Extracted mRNA may be subsequently detected by hybridization, such as Northern blot, and/or amplification, such as quantitative or semi-quantitative RT-PCR. Other methods of amplification include ligase chain reaction (LCR), transcription-mediated amplification (TMA), strand displacement amplification (SDA) and nucleic acid sequence-based amplification (NASBA).

Advantageously, the level of mRNA expression for each of the n genes may be measured by the mean of quantification of the cDNA synthesized from said mRNA, as a template, by one reverse transcriptase. Methods for determining the quantity of mRNA by microarrays or by RNA sequencing may also be used.

In certain embodiments, complexes between the double-stranded nucleic acids resulting from amplification and fluorescent SYBR® molecules may be obtained and then the fluorescence signal generated by the SYBR® molecules complexed with the said amplified nucleic acids may be measured.

The determination of the expression level of said n genes could be to carry out by a northern blot analysis, but due to the low efficiency of such a method, the skilled person will prefer the quantitative methods to obtain a more precise expression level of said n genes.

The group of 9 genes of the invention with the corresponding probe set and CDS (or one of the CDS if the gene expression different variants) are represented in Table 1.

TABLE 1

| | | | | Expression |
|---|---|---|---|---|
| | | | | level |
| | | | | of |
| Probe | Gene | CDS | β | reference |
| set | symbole | SEQ ID | coefficient | (ELR) |
| 235609_at | BRIP1 | SEQ ID NO:1 | 0.25527251 | 192 |
| 201241_at | DDX1 | SEQ ID NO:2 | 0.2787536 | 3182 |
| 201440_at | DDX23 | SEQ ID NO:3 | 0.23044892 | 1015 |
| 218481_at | EXOSC5 | SEQ ID NO:4 | 0.25527251 | 1412 |
| 242560_at | FANCD2 | SEQ ID NO:5 | 0.25527251 | 288 |
| 200594_x_{—} | HNRNPU | SEQ ID NO:6 | 0.32221929 | 11244 |
| at | | | | |
| 217786_at | PRMT5 | SEQ ID NO:7 | 0.23044892 | 1616 |
| 203182_s_{—} | SRPK2 | SEQ ID NO:8 | 0.32221929 | 966 |
| at | | | | |
| 223002_s_{—} | XRN2 | SEQ ID NO:9 | 0.32221929 | 767 |
| at | | | | |

### Step b)

The regression β coefficient reference values may be easily determined by the skilled man in the art for each gene of nucleic acid sequence SEQ ID NO:i using a Cox model. The Cox model is based on a modelling approach to the analysis of survival data. The purpose of the model is to simultaneously explore the effects of several variables on survival. The Cox model is a well-recognised statistical technique for analysing survival data. When it is used to analyse the survival of patients in a clinical trial, the model allows us to isolate the effects of treatment from the effects of other variables. The logrank test cannot be used to explore (and adjust for) the effects of several variables, such as age and disease duration, known to affect survival. Adjustment for variables that are known to affect survival may improve the precision with which the inventors can estimate the treatment effect. The regression method introduced by Cox is used to investigate several variables at a time. It is also known as proportional hazards regression analysis. Briefly, the procedure models or regresses the survival times (or more specifically, the so-called hazard function) on the explanatory variables. The hazard function is the probability that an individual will experience an event (for example, death) within a small-time interval, given that the individual has survived up to the beginning of the interval. It can therefore be interpreted as the risk of dying at time t. The quantity h0 (t) is the baseline or underlying hazard function and corresponds to the probability of dying (or reaching an event) when all the explanatory variables are zero. The baseline hazard function is analogous to the intercept in ordinary regression (since exp0=1). The regression coefficient β gives the proportional change that can be expected in the hazard, related to changes in the explanatory variables. The coefficient β is estimated by a statistical method called maximum likelihood. In survival analysis, the hazard ratio (HR) (Hazard Ratio=exp(β)) is the ratio of the hazard rates corresponding to the conditions described by two sets of explanatory variables. For example, in a drug study, the treated population may die at twice the rate per unit time as the control population. The hazard ratio would be 2, indicating higher hazard of death from the treatment.

In one embodiment, the regression β coefficient reference values are described in Table 1.

In the abovementioned formula, Ci=1 if the expression level of the gene of nucleic acid sequence SEQ ID NO:i is higher than an expression level of reference ELRi or Ci=−1 if the expression level of the gene of nucleic acid sequence SEQ ID NO:i is lower than or equal to ELRi, wherein “i” is as defined above for each DNA repair pathway group.

Expression level of reference ELRi may consist of “cut-off” values. A cut-off value is a value of expression of a gene that allows to separate the individuals according to their outcome (good or bad) for a given gene. If the measured expression value of the gene of an individual is higher than the cut-off value, the individual has a good outcome and vice-versa. The cut-off values may be obtained using Maxstat algorithm.

For example, each reference cut-off value ELRi for each gene may be determined by carrying out a method comprising the steps of:
a) providing a collection of samples from patients suffering from acute myeloid leukemia;
b) determining the expression level of the relevant gene for each sample contained in the collection provided at step a);
c) ranking the samples according to said expression level;
d) classifying said samples in pairs of subsets of increasing, respectively decreasing, number of members ranked according to their expression level;
e) providing, for each sample provided at step a), information relating to the actual clinical outcome for the corresponding cancer patient (i.e. the duration of the disease-free survival (DFS), or the event free survival (EFS), or the overall survival (OS) or both);
f) for each pair of subsets of tumour tissue samples, obtaining a Kaplan Meier percentage of survival curve;
g) for each pair of subsets of tumour tissue samples calculating the statistical significance (p value) between both subsets;
h) selecting as reference value ELR for the expression level, the value of expression level for which the p value is the smallest.

For example, the expression level of a gene has been assessed for 100 samples of 100 patients. The 100 samples are ranked according to the expression level of the gene. Sample 1 has the highest expression level and sample 100 has the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also, for each of the 99 groups, the p value between both subsets was calculated. The reference value ELRi is then selected such as the discrimination based on the criterion of the minimum p value is the strongest. In other terms, the expression level corresponding to the boundary between both subsets for which the p value is minimum is considered as the reference value. It should be noted that according to the experiments made by the inventors, the reference value ELRi is not necessarily the median value of expression levels.

In one embodiment, the ELRi are described in Table 1.

### Step c)

In case said individual has a score TRC_{score} higher than the reference value TRC_{ref}, said individual is likely to have an overall survival after 1000 days below 80%. By “below 80%”, it is meant in the invention 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% and 0%. In particular, the said individual is likely to have an overall survival after 1000 days below after 1500 days below 60%. Particularly, the said individual is likely to have an overall survival after 2500 days below 40%. Alternatively or additionally, the said individual is likely to have an event free survival after 1000 days below 50%.

In case said individual has a score TRC_{score} lower than the reference value TRC_{ref}, the said individual is likely to have an overall survival after 1000 days equal or over 80%. By “equal or over 80%”, it is meant in the invention 80%, 79%, 82%, 83%, 84%, 85%, 86%, 87%, 88%, 89%, 90%, 91%, 92%, 93%, 94%, 95%, 96%, 97%, 98%, 99% and 100%. In particular, the said individual is likely to have an overall survival after 1500 days over 80%. Particularly, the said individual is likely to have an overall survival after 2500 days equal or over 60%. By “equal or over 60%” it is meant in the invention 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%. Alternatively or additionally, the said individual is likely to have an event free survival after 1000 days equal or over 50%. By “equal or over 50%” it is meant in the invention 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%.

The invention also relates to a method for treating an individual afflicted by a multiple myeloma, the method comprising administering an effective amount of a composition comprising a, i.e., at least one, G-quadruplex (G4) stabilizer and/or a, i.e., at least one histone deacetylase (HDAC) inhibitor and/or a i.e., at least one bromodomain and extraterminal (BET) proteins inhibitor and/or a, i.e., at least one, nitrogen mustard.

The composition may comprise
solely a, i.e., at least one G4 stabilizer,
solely a, i.e., at least one HDAC inhibitor,
solely a, i.e., at least one BET proteins inhibitor,
solely at least one nitrogen mustard,
a, i.e., at least one G4 stabilizer and a, i.e., at least one HDAC inhibitor,
a, i.e., at least one G4 stabilizer and a, i.e., at least one BET proteins inhibitor,
at least one G4 stabilizer and at least one nitrogen mustard,
at least one G4 stabilizer and at least one HDAC inhibitor and at least one nitrogen mustard,
at least one G4 stabilizer and at least one BET proteins inhibitor and at least one nitrogen mustard,
a, i.e., at least one HDAC inhibitor and a, i.e., at least one BET protein inhibitor,
a, i.e., at least one G4 stabilizer and a, i.e., at least one HDAC inhibitor and a, i.e., at least one BET proteins inhibitor, or
at least one G4 stabilizer and at least one HDAC inhibitor and at least one BET protein inhibitor and at least one nitrogen mustard.

The term “effective amount”, also referred to herein as “dosage”, refers to an amount of the composition that is suitable to treat a multiple myeloma in an individual.

In particular, the said method comprises the following steps:
a) identifying the patient able to respond to a composition comprising at least one G-quadruplex (G4) stabilizer and/or at least one histone deacetylase inhibitor and/or at least one at least one bromodomain and extraterminal (BET) proteins inhibitor and/or at least one nitrogen mustard by the following sub steps:
   i) measuring in a biological sample from said individual, the expression level of n genes, wherein n is an integer from 3 to 9 and wherein the n genes are selected from a group of 9 genes consisting of the nucleic acid sequences SEQ ID NO:1 to SEQ ID NO:9;
   ii) calculating a score TRC_{score} according to the following formula

$$T R {C}_{s c o r e} = {\sum }_{i = 1}^{n} β i \times C i$$

wherein βi represents the regression β coefficient reference value of one of the n genes selected at step a), wherein Ci=1 if the expression level of the said gene is higher than an expression level of reference and Ci=−1 if the expression level of the said gene is lower than or equal to ELRi,
iii) confirming that the said individual has a score TCR_{score} higher than a reference value TRC_{ref},
iv) concluding that the individual is able to respond a composition comprising at least one G-quadruplex (G4) stabilizer and/or at least one histone deacetylase inhibitor and/or at least one at least one bromodomain and extraterminal (BET) proteins inhibitor,
b) administering to the individual the composition comprising at least one G-quadruplex (G4) stabilizer and/or at least one histone deacetylase inhibitor and/or at least one at least one bromodomain and extraterminal (BET) proteins inhibitor.

In particular, the said method comprises the following steps:
a) identifying the patient able to respond to a composition comprising at least one G-quadruplex (G4) stabilizer and/or at least one histone deacetylase inhibitor and/or at least one at least one bromodomain and extraterminal (BET) proteins inhibitor and/or at least one nitrogen mustard by the following sub steps:
   i) measuring in a biological sample from said individual, the expression level of 9 genes consisting of the nucleic acid sequences SEQ ID NO:1 to SEQ ID NO:9;
   ii) calculating a score TRC_{score} according to the following formula

$$T R {C}_{s c o r e} = {\sum }_{i = 1}^{9} β i \times C i$$

wherein βi represents the regression β coefficient reference value for the gene of nucleic acid sequence SEQ ID NO:i,
wherein Ci=1 if the expression level of the gene of nucleic acid sequence SEQ ID NO:i is higher than an expression level of reference Ci=−1 if the expression level of the gene of nucleic acid sequence SEQ ID NO:i is lower than or equal to ELRi,
iii) confirming that the said individual has a score TCR_{score} higher than a reference value TRC_{ref}, wherein the reference value TRC_{ref} is of −0.39535,
iv) concluding that the individual is able to respond a composition comprising at least one G-quadruplex (G4) stabilizer and/or at least one histone deacetylase inhibitor and/or at least one at least one bromodomain and extraterminal (BET) proteins inhibitor,
b) administering to the individual the composition comprising at least one G-quadruplex (G4) stabilizer and/or at least one histone deacetylase inhibitor and/or at least one at least one bromodomain and extraterminal (BET) proteins inhibitor.

The invention also relates to the use of a composition comprising at least one HDACi and at least one BET inhibitor for its use in the treatment of an individual afflicted by a multiple myeloma.

Advantageously, the at least one HDACi and at least one at least one BET inhibitor are used simultaneously, separately, or sequentially.

Advantageously, the at least one HDACi and at least one BET proteins inhibitor are associated with a pharmaceutical acceptable vehicle. The pharmaceutical acceptable vehicle is as defined above.

It is within the skills of a physician to determine the specific therapeutically effective dosage regimen, as this dosage regimen will be dependent upon a variety of factors including, but not limited to: the severity of the multiple myeloma; the age; the body weight; general health; the sex; the diet; the time course of administration; the route of administration; the duration of the treatment; the drugs that are concomitantly administered in combination with the pharmaceutical composition within the scope of the present invention.

In some embodiments, the dosage regimen of each of said at least one HDACi and at least one BET proteins inhibitor ranges from about 0.0001 mg to about 1,000 mg per adult, per day. Preferably, the individual is administered with an amount of about 0.0001, 0.0005, 0.001, 0.005, 0.01, 0.05, 0.1, 0.5, 1.0, 2.5, 5.0, 7.5, 10.0, 15.0, 20.0, 25.0, 50.0, 75.0, 15 100, 250, 500 and 750 mg of each of said at least one G4 stabilizer and at least one histone deacetylase inhibitor in order to adjust the dosage regimen that is the most suitable to a particular individual in need of the treatment.

A pharmaceutical composition within the scope of the present invention may contain from about 0.01 mg to about 500 mg of each of the said at least one G4 stabilizer and at least one histone deacetylase inhibitor, preferably from about 1 mg to about 100 mg of said at least one G4 stabilizer and at least one histone deacetylase inhibitor.

In a preferred embodiment, an effective amount of each of the said at least one HDACi and at least one BET proteins inhibitor is routinely administered to an individual in need thereof, at a dosage regimen from about 0.0002 mg/kg to about 20 mg/kg of body weight per day, in particular from about 0.001 mg/kg to 7 mg/kg of body weight per day.

The optimal amount of the said at least one HDACi and at least one BET proteins inhibitor to be comprised in a pharmaceutical dosage unit according to the invention may be easily adapted by the one skilled in the art using routine known protocols or methods.

Said composition may be administered by any suitable route, i.e. including, but not limited to, an oral, sublingual, buccal, subcutaneous, transdermal, topical, intraperitoneal, intramuscular, intravenous, subdermal, intrathecal and intranasal and rectal administration.

The invention further relates to a pharmaceutical composition comprising at least one G-quadruplex (G4) stabilizer and at least one histone deacetylase inhibitor and/or at least one bromodomain and extraterminal (BET) proteins inhibitor and/or at least one nitrogen mustard.

The invention also relates to a pharmaceutical composition comprising at least one histone deacetylase inhibitor and at least one bromodomain and extraterminal (BET) proteins inhibitor.

The invention further relates to a method for prognosing the outcome of an individual afflicted by a multiple myeloma, wherein said method comprises the following steps:
a) measuring in a biological sample from said individual, the expression level of n genes, wherein n is an integer from 3 to 9 and wherein the n genes are selected from a group of 9 genes consisting of the nucleic acid sequences SEQ ID NO:1 to SEQ ID NO:9;
b) calculating a score TRC_{score} according to the following formula

$$T R {C}_{s c o r e} = {\sum }_{i = 1}^{n} β i \times C i$$

wherein βi represents the regression β coefficient reference value of one of the n genes selected at step a), wherein Ci=1 if the expression level of the said gene is higher than an expression level of reference and Ci=−1 if the expression level of the said gene is lower than or equal to ELRi,
c) prognosing that
   said individual with a score TCR_{score} higher than a reference value TRC_{ref} is likely to have a bad outcome, or
   said individual with a score TCR_{score} lower than a reference value TRC_{ref} is likely to have a good outcome.

The different combinations of n genes from the group of 9 genes of the invention are listed in Table 2 above.

In particular, n equal to 9 and the reference value TRC_{ref} is of −0.39535.

The invention also relates to a composition comprising at least one G-quadruplex (G4) stabilizer and/or at least one histone deacetylase inhibitor and/or at least one at least one bromodomain and extraterminal (BET) proteins inhibitor and/or at least one nitrogen mustard for its use in a method for treating an individual afflicted by multiple myeloma, wherein the individual has been prognosed with a poor outcome according to the above-mentioned method.

### LEGENDS TO THE FIGURES

FIG. 1 represents the expression of R-loops and TRC resolution genes for the different stage of Memory B cell (MBC) to plasma cell (PC) differentiation. The genes significantly overexpressed in PrePBs compared to MBCs, PBs and PCs were determined with a SAM (Significance Analysis of Microarrays) multiclass analysis (False discovery rate (FDR)=0), identifying 41 unique genes. An unsupervised hierarchical clustering was completed and the normalized expression value for each gene is indicated in shade of grey, with black representing high expression and white representing low expression. MBC1 to 5 represent 5 different samples of memory B cells. PrePB1 to 5 represent 5 different samples of preplasmablasts. PB1 to 5 represents 5 different samples of plasmablasts, PC1 to 5 represent 5 different samples of Plasma cells.

FIG. 2 represents Kaplan Meier curves showing the percentage of survival of the patients of the training cohort vs time (days) based on different genes' expression. In all graphs, curve 1 represents low gene expression results and curve 2 represents high expression results. The gene expression prognostic value was determined using Maxstat R algorithm. FIG. 1A represents the results obtained for BRIP1 expression, wherein for curve 1 n=92, for curve 2 n=253, p-value=0.0012 and hazard ratio (HR)=1.8. FIG. 1B represents the results obtained for DDX1 expression, wherein for curve 1 n=264, for curve 2 n=81, p-value=0.0011 and HR=1.9. FIG. 1C represents the results obtained for DDX23, wherein for curve 1 n=304, for curve 2 n=41, p-value=0.044 and HR=1.7. FIG. 1D represents the results obtained for EXOSC5 expression, wherein for curve 1 n=259, for curve 2 n=86, p-value=0.0019 and HR=1.8. FIG. 1E represents the results obtained for FANCD2 expression, wherein for curve 1 n=168, for curve 2 n=177, p-value=0.0037 and HR=1.8.

FIG. 3 represents Kaplan Meier curves showing the percentage of survival of the patients of the training cohort vs time (days) based on different genes' expression. In all graphs, curve 1 represents low gene expression results and curve 2 represents high expression results. The gene expression prognostic value was determined using Maxstat R algorithm. FIG. 3A represents the results obtained for HNRNPU expression, wherein for curve 1 n=304, for curve 2 n=41, p-value=0.0014 and HR=2.1. FIG. 3B represents the results obtained for PRMT5 expression, wherein for curve 1 n=268, for curve 2 n=77, p-value=0.013 and HR=1.7. FIG. 3C represents the results obtained for SRPK2 expression, wherein for curve 1 n=87, for curve 2 n=258, p-value=0.0031 and HR=2.1. FIG. 3D represents the results obtained for XRN2 expression, wherein for curve 1 n=298, for curve 2 n=47, p-value=0.0014 and HR=2.1.

FIGS. 4A and 4C represents Kaplan Meier curves showing the percentage of event free survival of the patients of the training cohort (FIG. 4A) or of the validation cohort (FIG. 4C) vs time (days) based on the TRC_{score}. FIGS. 4B and 4D represents Kaplan Meier curves showing the percentage of survival of the patients of the training cohort (FIG. 4A) or of the validation cohort (FIG. 4C) vs time (days) based on the TRC_{score}. Patients of the different cohort were split in two groups: high risk patients (n=119 for the training cohort; n=71 for the validation cohort) and low risk patients (n=226 for the training cohort; n=135 for the validation cohort). High risk patients have a TRC_{score} higher than TRC_{ref} and low risk patients have a TRC_{score} lower or equal to TRC_{ref}. In all graphs, curve 1 represents the results for high-risk patients, and curve 2 represents the results for low risk patients. The parameters to compute the TRCs score of patients in the HM cohort and the proportions delineating the two prognostic groups were those defined with the TT2 cohort.

FIG. 5A represents the IC50 of Panobinostat (nM) as function of the TRC_{score} of 11 Huma Myeloma Cell Lines (HMCLs): JJN3, OPM2, RPM18266, LP1, AMO1, SKMM2, XG1, XG2, XG6, XG7, XG12, XG19, XG20 and XG21. The represented best-fit line has a r=−0.55 with a p-value=0.05. FIG. 5B represents tumor cell viability as function of the TRC_{score} of 12 primary myeloma cells. The represented best-fit line has a r=−0.67 with a p-value lower than 0.01. Mononuclear cells from tumour samples of 12 patients with MM were cultured for 4 days in the presence of IL6 (2 ng/mL) with or without increasing concentrations (0 nM, 0.6 nM, 1.25 nM, 2.5 nM, 5 nM, 10 nM and 20 nM) of Panobinostat. At day 4 of culture, the count of viable MM cells was determined using CD138 and CD38 staining by flow cytometry.

FIG. 6A represents the IC50 of Pyridostatin (nM) for 11 HMCLs: a is OPM2; b is XG12; c is XG21; d is XG19; e is RPM18266; f is LP1; g is XG6; h is JJN3; I is XG2; j is XG7; k: L363; I is XG1; m is AMO1; n is SKMM2 and o is XG20. IC50 of Pyridostatin +/−SD are representative of 3 independent experiments. NA: not reached. FIG. 6B represents the Pyridostatin toxicity on bone marrow cells (x-axis is the Pyridostatin concentration in μM; y-axis is the cell count in percentage of the control (0 μM of Pyridostatin)). The toxicity of Pyridostatin was assessed on primary bone marrow cells from one MM patient (dark grey bars), co-cultured with normal bone marrow microenvironment (light grey). The toxicity on MM cells and normal bone marrow cells was assessed by flow cytometry using CD138 and CD28 marker cells. FIG. 6C represents the Pyridostatin toxicity on bone marrow cells (x-axis is the Pyridostatin concentration in μM; y-axis is the cell count in percentage of the control (0 μM of Pyridostatin)). The toxicity of Pyridostatin was assessed on primary bone marrow cells from five MM patient (black bars) and co-cultured with normal bone marrow microenvironment (grey bars). The toxicity on MM cells and normal bone marrow cells was assessed by flow cytometry using CD138 and CD28 marker cells. Data are mean values of five independent experiments. P-value: *<0.05; **<0.01; ***<0.001 using a student t-test for pairs.

FIGS. 7A and 7B represent Western blots. Doxy stands for doxycycline. PDS stands for Pyridostatin (concentration in μM). XG7 cell line was transduced with a doxycycline-inducible lentivirus containing RNase H gene (XG7-RH). XG7-RH was cultured with or without doxycycline in absence or presence of Pyridostatin during 5 h or 24 h. Western blot membranes were stained with anti-Phospho-Chk2, anti-Phospho-P53, anti-P53, anti-PARP, anti-myc (FIG. 7A) and anti-p21, anti-p16, anti-p27 and anti-gH2AX (FIG. 7B). Tubulin protein level was used as control.

FIGS. 8A and 8B represent the IC50 of a bromodomain and extraterminal proteins inhibitor (FIG. 8A: I-BET-762; FIG. 8B: RVX-208) as function of the TRC_{score} of 11 Human Myeloma Cell Lines (HMCLs): JJN3, OPM2, RPM18266, LP1, AMO1, SKMM2, XG1, XG2, XG6, XG7, XG12, XG19, XG20 and XG21. For FIG. 8A, the represented best-fit line has a r=−0.66 with a p-value<0.05. For FIG. 8B, the represented best-fit line has a r=−0.88 with a p-value<0.05. HMCLs with high TRC score are significantly more sensitive to I-BET-762 and RVX-208 bromodomain and extraterminal proteins inhibitors treatment compared to cell lines with low TRC score.

FIGS. 9A and 9B represent cell viability of two HMCLs (9A: JJN3; 9B: XG7) treated with increasing concentration of Pyridostatin (x-axis in μM) and Panobinostat (y-axis in μM) for 4 days. The synergistic combination is represented in shade of grey (arbitrary unit), with black representing high synergism and white representing high antagonism.

FIG. 10 represents cell viability of XG2 cell line treated with increasing concentration of Pyridostatin (x-axis in μM) and Panobinostat (y-axis in μM) for 4 days. The synergistic combination is represented in shade of grey (arbitrary unit), with black representing high synergism and white representing high antagonism.

FIG. 11 represents the toxicity of Pyridostatin alone, Panobinostat alone and the combination of Pyridostatin and Panobinostat on bone marrow cells. X-axis is the concentration in μM of the drug: 0: control; A: Pyridostatin at 1.1; B: Panobinostat at 2.5; C: Pyridostatin at 1.1 and Panobinostat at 2.5.Y-axis is the cell count in percentage of the control (0 μM of drug)). The toxicity was assessed on primary bone marrow cells from one MM patient cultured alone (black bars) and co-cultured with normal bone marrow microenvironment (grey bars). The toxicity on MM cells and normal bone marrow cells was assessed by flow cytometry using CD138 and CD28 marker cells.

FIG. 12 represents cell viability of XG7 cell line treated with increasing concentration of Pyridostatin (x-axis in μM) and I-BET-762 (y-axis in μM) for 4 days. The synergistic combination is represented in shade of grey (arbitrary unit), with black representing high synergism and white representing high antagonism.

FIG. 13 represents cell viability of XG1 cell line (A) and XG2 cell line (B) treated with increasing concentration of Pyridostatin (x-axis in μM) and Melphan (y-axis in μM) for 4 days. The synergistic combination is represented in shade of grey (arbitrary unit), with black representing high synergism and white representing high antagonism.

FIG. 14 represents cell viability of XG2 cell line (A) and XG2 Melphalan-resistant (MeIR) cell line (B) treated with increasing concentration of Pyridostatin (x-axis in μM) and Melphan (y-axis in μM) for 4 days. The synergistic combination is represented in shade of grey (arbitrary unit), with black representing high synergism and white representing high antagonism.

FIG. 15 represents Melphalan IC50 on XG2 (A) and XG2-MeIR (B) cell lines. Grey bars relate to the respective myeloma cells treated with increasing concentration of Melphan (y-axis in μM). For the XG2 cell line, Melphalan IC50 was 0.6325 μM. For the XG2 cell line, Melphalan IC50 was 0.35 μM. Black bars relate to the respective myeloma cells treated with increasing concentration of Melphalan (y-axis in μM) and Pyridostatin at 1.25 μM for 4 days. For the XG2 cell line, Melphalan IC50 was 1.4875 μM. For the XG2 cell line, Melphalan IC50 was 0.6325 μM. P-value: **<0.01; ***<0.001 using a student t-test for pairs. “ns” stands for non-significant.

FIG. 16 relates to DDX23 depletion in XG7 cells. Two groups of XG7 cells were transduced with a doxycycline-inducible lentivirus containing an shRNA targeting DDX23 giving XG7-shDDX23-1 group cell and XG7-shDDX23-2 group cell. Each group cell was cultured without or with doxycycline for 24 or 48 hours. Protein detection was assayed using western blot analysis (A). Membranes were stained with anti-PARP, anti-DDX23, anti-γH2AX and anti-tubulin. Tubulin protein level was used as control for assaying DDX23 protein level (B). Y-axis on FIG. 17B represents the ratio of DDX23 level on tubulin level. DDX23 mRNA level was assayed by RT-qPCR (C). On FIGS. 16B and 16C, black bars relate to XG7-shDDX23-1 group cell, and black bar relate to XG7-shDDX23-2 group cell. P-value: *<0.05; **<0.01; ***<0.001 using a student t-test for pairs. “ns” stands for non-significant.

FIG. 17 relates to DDX1 depletion in XG7 cells. Two groups of XG7 cells were transduced with a doxycycline-inducible lentivirus containing an shRNA targeting DDX1 giving XG7-shDDX1-4 group cell and XG7-shDDX1-5 group cell. Each group cell was cultured without or with doxycycline for 24 or 48 hours. Protein detection was assayed using western blot analysis (A). Membranes were stained with anti-PARP, anti-DDX23, anti-γH2AX and anti-tubulin. Tubulin protein level was used as control for assaying DDX23 protein level (B). Y-axis on FIG. 17B represents the ratio of DDX1 level on tubulin level. DDX1 mRNA level was assayed by RT-qPCR (C). On FIGS. 16B and 16C, grey bars relate to XG7-shDDX1-4 group cell, and black bar relate to XG7-shDDX1-5 group cell. P-value: *<0.05; **<0.01; ***<0.001 using a student t-test for pairs. “ns” stands for non-significant.

FIG. 18 represents cell viability of XG7 cells depleted for DDX23. XG7-shDDX23-1 and XG7-shDDX23-2 group cells were exposed to doxycycline and cell viability was analyzed by trypan blue assay. Results are those of three independent experiments. FIG. 18A is a diagram representing XG7-shDDX23-1 group cell viability after 2, 3 or 6 days of culture without (control in grey bars) or with (black bars) doxycycline at 1 μg/ml. Y-axis represents the ratio of cells relatively to the control. FIG. 18B is a diagram representing XG7-shDDX23-2 group cell viability after 2, 3 or 6 days of culture without (control in grey bars) or with (black bars) doxycycline at 1 μg/ml. Y-axis represents the ratio of cells relatively to the control. FIG. 18C represents the number of cells after 0, 2, 3 or 6 days of culture without or with doxycycline at 1 μg/ml (1: XG7-shDDX23-1 without doxycycline; 2: XG7-shDDX23-1 with doxycycline; 3: XG7-shDDX23-2 without doxycycline; 4: XG7-shDDX23-2 with doxycycline). Cell count was analyzed by trypan blue assay. Results are mean of three independent experiments.

FIG. 19 represents cell viability of XG7 cells depleted for DDX1. XG7-shDDX1-4 and XG7-shDDX1-5 group cells were exposed to doxycycline and cell viability was analyzed by trypan blue assay. Results are those of three independent experiments. FIG. 19A is a diagram representing XG7-shDDX1-4 group cell viability after 2, 3 or 6 days of culture without (control in grey bars) or with (black bars) doxycycline at 1 μg/ml. Y-axis represents the ratio of cells relatively to the control. FIG. 19B is a diagram representing XG7-shDDX1-5 group cell viability after 2, 3 or 6 days of culture without (control in grey bars) or with (black bars) doxycycline at 1 μg/ml. Y-axis represents the ratio of cells relatively to the control. FIG. 19C represents the number of cells after 0, 2, 3 or 6 days of culture without or with doxycycline at 1 μg/ml (1: XG7-shDDX1-4 without doxycycline; 2: XG7-shDDX1-4 with doxycycline; 3: XG7-shDDX1-5 without doxycycline; 4: XG7-shDDX1-5 with doxycycline). Cell count was analyzed by trypan blue assay. Results are mean of three independent experiments.

### EXAMPLE

### 1. Patients and Method

### Cell Lines

JJN3, OPM2, RPM18266, LP1, AMO1, SKMM2 human myeloma cell lines (HMCLs) were obtained from ATCC (Molshein, France) and were cultured in RPMI 1640 medium, supplemented with 10% fetal calf serum (Biochrom, Berlin, Germany) and 2 mM L-Glutamine. The interleukin-6-dependent cell lines XG1, XG2, XG6, XG7, XG12, XG19, XG20, XG21 were obtained as previously described and maintained in the presence of 2 ng/mL recombinant IL-6 (R&D Systems, Oxon, UK), 10% fetal calf serum and 2 mM L-Glutamine.

### Gene Expression Data

Affymetrix data from two independent cohorts of previously untreated patients with MM were used. These data are publicly available through the ArrayExpress database (E-MTAB-372). The training cohort (TT2 cohort) included 345 patients with MM from the University of Arkansas for Medical Sciences (UAMS, Little Rock, AR, USA). These data can be accessed at the online Gene Expression Omnibus (GSE2658). The validation cohort consisted of 206 patients with MM and was called Heidelberg-Montpellier (HM) cohort. This cohort also included five BMPC samples from healthy donors. Samples were obtained after signature of a written informed consent form in accordance with the Declaration of Helsinki and after approval by the Ethics Committees of Montpellier (DC-2008-417) and Heidelberg. After Ficoll-density gradient centrifugation, plasma cells were purified using anti-CD138 MACS microbeads (Miltenyi Biotech, Bergisch Gladbach, Germany). The clinical characteristics of the two cohorts have been previously described (Moreaux, J. et al, Mol. Cancer Ther. 11, 2685-2692 (2012) and Hose, D. et al., Haematologica 96, 87-95 (2011).

Affymetrix data from another cohort of patient treated with Daratumumab, the Daratumumab cohort, was also used. The clinical characteristics of this cohort have been described in Pochard et al., Integrative approach to find predictive markers of response to daratumumab in multiple myeloma, EHA Library, 2021.

### Selection of Prognostic Genes

To establish gene expression (GE)-based risk scores, the inventors selected probe sets whose expression values were significantly associated with overall survival, using MaxStat R function and Benjamini Hochberg multiple testing correction (adjusted p-value <0.05).

### Building Gene Expression-Based Risk Score (TCR_{Score}) on Training Cohort

The GE-based risk score was built as the sum of the beta coefficients weighted by +1 or −1 according to the patient signal above or below/equal the probe set MaxStat value. Patients from the training cohort were ranked according to increased prognostic score and for a given score value X, the difference in survival of patients with a prognostic score ≤X or >X was computed using MaxStat analysis. Survival analyses were assessed using Kaplan-Meier method, and survival curves were compared using log-rank test.

The build TCRscore was validated with the validation cohort, using the cutoff values determined for the training cohort. Survival analyses were assessed using Kaplan-Meier method, and survival curves were compared using log-rank test.

### Patient Samples

All primary samples were collected with the approval of the institutional research board from Montpellier University hospital (DC2008-417) and in accordance with the Declaration of Helsinki. Patient's written informed consent was obtained. Mononuclear cells were isolated from total BM using Ficoll density gradient centrifugation (Nycomed, Zurich, Switzerland) and cultured in the presence of 1 ng/mL IL-6. Four days after treatment with increased concentrations of panobinostat or pyridostatin, the percentage CD138+ viable plasma cells and CD138− viable non-myeloma cells was determined by flow cytometry.

### Compounds

Panobinostat, pyridostatin and I-BET-762 were obtained from Sellekchem (Munich, Germany). For in vitro studies, pyridostatin was dissolved in water, panobinostat and I-BET-762 in dimethyl sulfoxide (DMSO). Aliquots were stored at −20° C.

### Cell Count and Cell Viability

Cells in culture were stained using trypan blue solution (Sigma-Aldrich, St. Louis MO, USA) and viable cells were counted under a light microscope. The CellTiter-Glo Luminescent Viability assay (Promega, Leiden, The Netherlands) was used to measure the effect on cell viability according to manufacturer's instructions, and the 50% inhibition (IC50) was determined using GraphPad Prism software (http://www.graphpad.com/scientific-software/prism/).

### Drug Combination Study

The interaction between pyridostatin and panobinostat, pyridostatin and I-BET-762 and pyridostatin and Melphan was investigated with a concentration matrix test, in which increasing concentration of each single drug were assessed with all possible combinations of the other drugs. Cell viability was measured using ATP quantification to obtain the viability matrix. For each combination, the percentage of expected growing cells in case of effect independence was calculated according to the Bliss equation: fuC=fuA, fuB, where fuC is the expected fraction of cells unaffected by the drug combination in the case of effect independence, and fuA and fuB are the fractions of cells unaffected by treatment A and B, respectively. The difference between the fraction of living cells in the cytotoxicity test and the fuC value was considered as an estimation of the interaction effect, with positive values indicating synergism and negative values antagonism.

Western Blot Analysis Western blot analysis was performed as previously described (Viziteu, E. et al., Leukemia 31, 2104-2113, 2017). Tubulin was used as loading control. All antibodies used were purchased from Cell Signaling, except for p21, p27, Myc, and phospho-Histone H2A.X. Myc antibody was obtained from Santa Cruz biotechnology (Texas, USA), phospho-Histone H2A.X antibody from Merck Millipore (Sigma-Aldrich), and p27 antibody was from Sigma-Aldrich.

### Statistical Analysis

Statistical analysis was performed using GenomicScape web tool (http://www.genomicscape.com). A Mann-Whitney U test and 1-way analysis of variance was used to compare 2 groups or multiple groups, respectively. GEP data were normalized with MAS5 algorithm and analyzed with GenomicScape and R and Bioconductor programs. Probe sets were selected for prognostic significance using the Maxstat R function and the Benjamini Hochberg multiple testing correction. The difference in OS was assayed with a log-rank test, and survival curves were plotted using the Kaplan-Meier method. The prognostic value of BRIP1, DDX1, DDX23, EXOSC5, FANCD2, HNRNPU, PRMT5, SRPK2 and XRN2 was computed using a maximally selected rank test from the R package maxstat, which allowed us to determine the optimal cut point for continuous variables.

### Inducible Depletion of DDX1 and DDX23

XG7 cells were transduced with shDDX1 or shDDX23 lentiviruses and stable transduced cells (XG7-shDDX23-1; XG7-shDDX23-2; XG7shDDX1-4; XG7-shDDX1-5) were obtained by adding 10 μg/ml puromycin. The expression of the shRNAs was induced by adding Doxycycline (1 ug/ml) 24 h after plating the cells.

### RT-qPCR

Total RNA was extracted using the RNeasy Kit (Qiagen) and reverse transcribed with the Reverse Transcription Kit (Qiagen). The measurement of gene expression was performed using the Roche LC480 Sequence Detection System. For each primer, serial dilutions of standard cDNA were amplified to create a standard curve, and values of unknown samples were estimated relative to this standard curve in order to assess PCR efficiency. Ct values were obtained for rRNA 18S and the respective genes of interest during the log phase of the cycle. Gene expression was normalized to that of rRNA 18s (ΔCt=Ct gene of interest-Ct 18s) and compared with the values obtained for a known positive control using the following formula: 100/2ΔΔCt where ΔΔCt=ΔCt unknown −ΔCt positive control.

### 2. Results

Multiple myeloma is clinically and biologically heterogeneous with several genetic alterations proposed as driving events in myelomagenesis, which are associated with growth advantage and cell cycle deregulation. Malignant PCs continue to produce elevated levels of immunoglobulins, underlying that transcription is highly active in those cells. Like non-transformed plasma cells, myeloma cells contain a well-developed endoplasmic reticulum (ER) and Golgi complex tailored to produce and secrete large amounts of immunoglobulins, underlying their characteristic morphology.

Besides, during normal PC differentiation, MBCs differentiate into pre-plasmablasts (prePBs), plasmablasts (PBs), early PCs, and finally, long-lived PCs that produce high Ig amounts. the pre-plasmablastic stage is associated with high proliferation following B cell activation (50% of cells in S-phase) and the start of Ig secretion. This led the inventors to hypothesis that conflicts between replication and transcription should be tightly managed at this specific stage to avoid tumorigenesis. Then, the inventors investigate the TRCs levels in malignant PCs.

### Probe Sets Selection

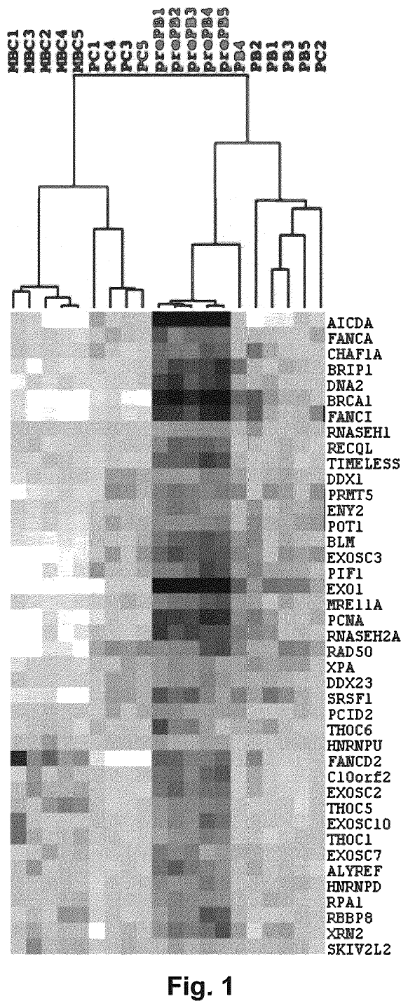

The inventors first aimed to identify genes involved in TRCs resolution significantly overexpressed in prePBs during B to PC differentiation. A list set of 83 genes involved in TRCs resolution was defined using the review of the literature. In that purpose, the inventors used their own in vitro model of B to PC differentiation. Indeed, the inventors have shown that PC generation can be modeled using multi-step culture systems to reproduce the sequential cell differentiation occurring in the different organs/tissues in vivo. In this model, memory B cells (MBCs) differentiate into pre-plasmablasts (prePBs), plasmablasts (PBs), early PCs and, finally, into long-lived PCs (LLPCs) (Jourdan M, Caraux A, Caron G, Robert N, Fiol G, Reme T, et al., J Immunol 2011; 187:3931-41; Jourdan M, CarauxA, De Vos J, Fiol G, Larroque M, Cognot C, et al., Blood 2009; 114:5173-81 and Jourdan M, Cren M, Robert N, Bollore K, Fest T, Duperray C, et al., Leukemia 2014, August; 28(8):1647-56). Using Significance Analysis of Microarrays (SAM) multi-class analysis, the inventors found 41 genes significantly overexpressed in prePBs compared to MBCs, PBs and PCs with a false discovery rate <5% (FIG. 1). These results confirmed the hypothesis of the inventors that the TRCs resolution is highly relevant in prePBs.

Considering the potent role of TRCs resolution in MM cell adaptation to replication stress, the inventors sought to identify the TRCs resolution factors that are associated with a poor outcome in MM. They first compared the Affymetrix gene expression profiles of purified normal bone marrow PCs (n=5) and purified MM cells from newly diagnosed patients (n=206) using the GenomicScape webtool (Kassambara, A. et al., PLoS Comput. Biol. 11, (2015) showed that 13 TRC resolution factors were significantly overexpressed in MM cell samples (fold change 2, false discovery rate <5%; 1000 permutations) (Data not shown).

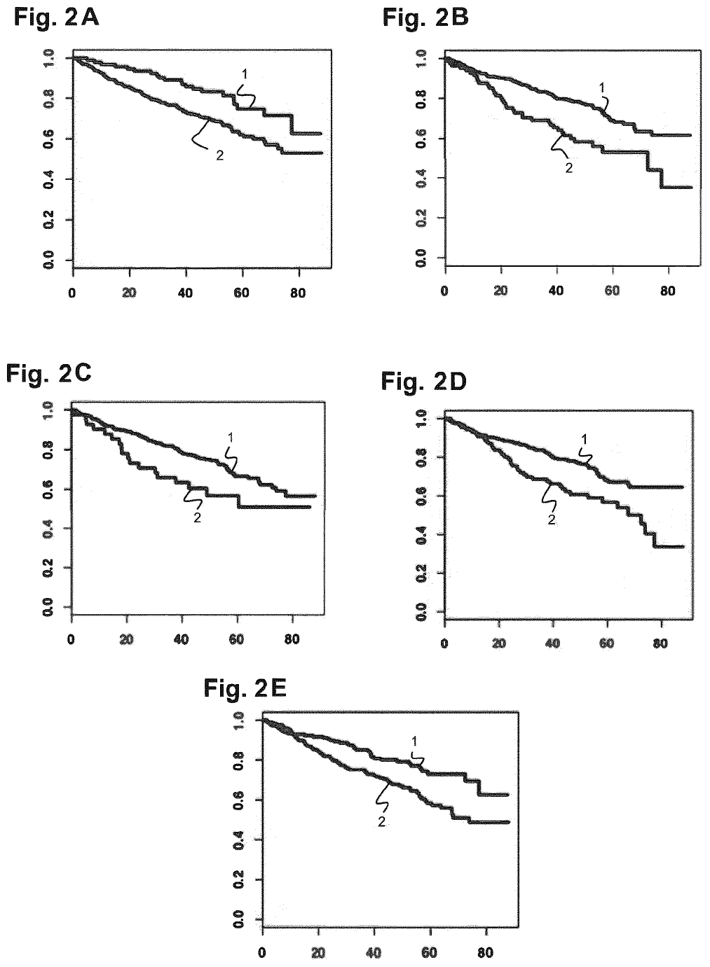

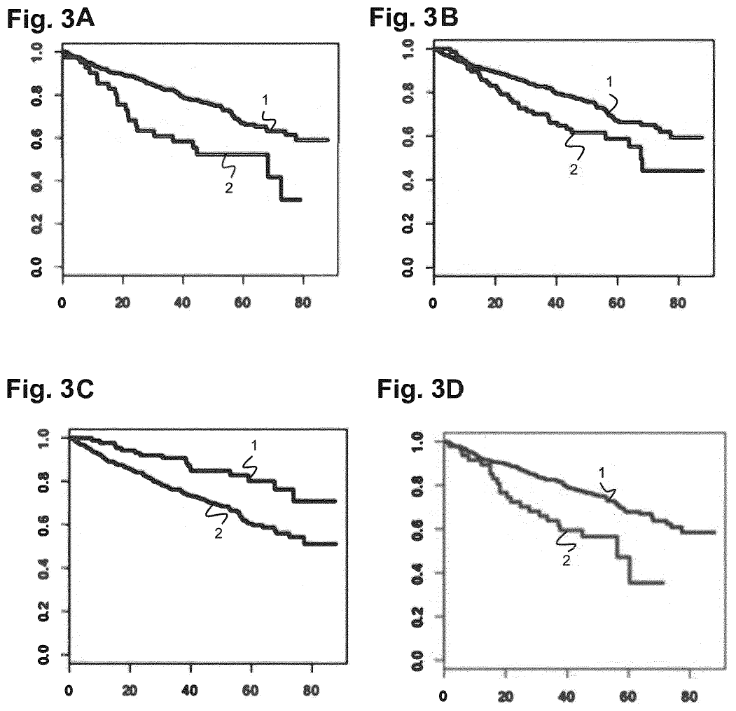

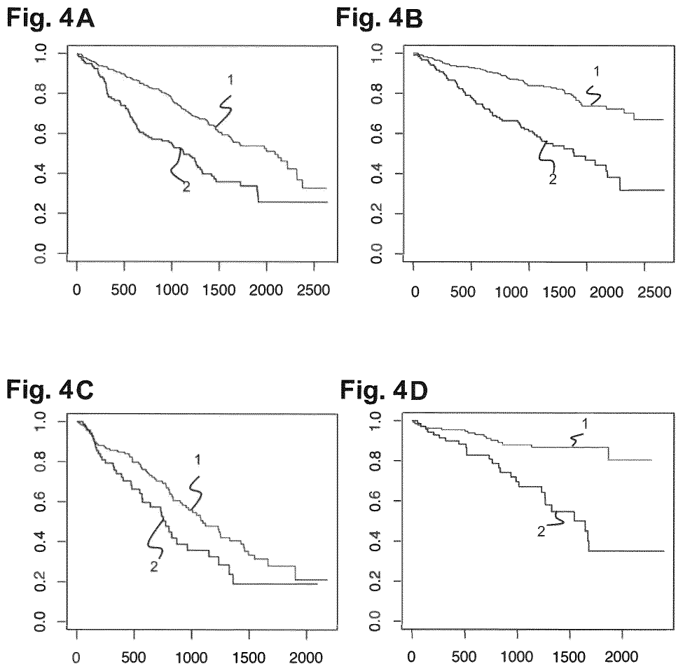

Then, the inventors analysed the publicly available Affymetrix gene expression profiles (Gene Expression Omnibus, GSE2658) of the training cohort with the MaxStat R function and found out a high expression of 9 of these 13 TRC resolution factors was associated with poor MM outcome (FIGS. 2 and 3). This result suggested to the inventors that increasing TRCs could be a specific therapeutic strategy to kill malignant plasma cells. In that objective, the inventors gathered the prognostic value of these 9 genes within a GEP-based TRC resolution score (TRC_{score}). High TRC_{score} has been identified associated with a poor outcome (EFS and OS) in two independent cohorts (training and validation cohorts) of newly diagnosed MM patients treated by high dose therapy and autologous stem cell transplantation (FIG. 4).

### HDAC Inhibitors' Sensitivity

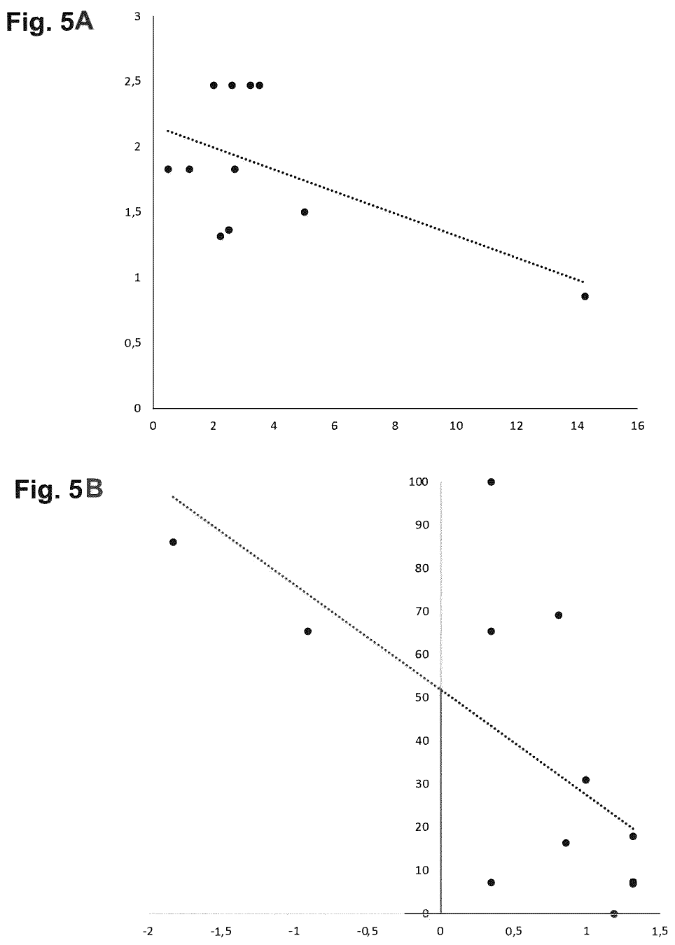

By investigating the link between the TRC score and the response of MM cells to drugs using their own collection of human myeloma cell lines (HMCL), the inventors identified that HMCLs with high TRC score values are significantly more sensitive to Panobinostat histone deacetylase (HDAC) inhibitor, currently used in MM treatment at relapse (FIG. 5A). Without being bound by any theory, the inventors suggest that this result could be explained by that during chromatin opening, acetylation promotes R-loop formation that will then constitute obstacles to replication fork progression.

Furthermore, the inventors validated these results using primary MM cells from patients (n=12) co-cultured with their bone marrow microenvironment (FIG. 5B). A high TRC score value is associated with a higher toxicity of Panobinostat in primary MM cells. This underlines that the TRC score allows the identification of a subgroup of MM patients with a poor outcome that could benefit from Panobinostat HDACi treatment.

### G-Quadruplex (G4) Stabilizers' Sensitivity in MM Cell Lines

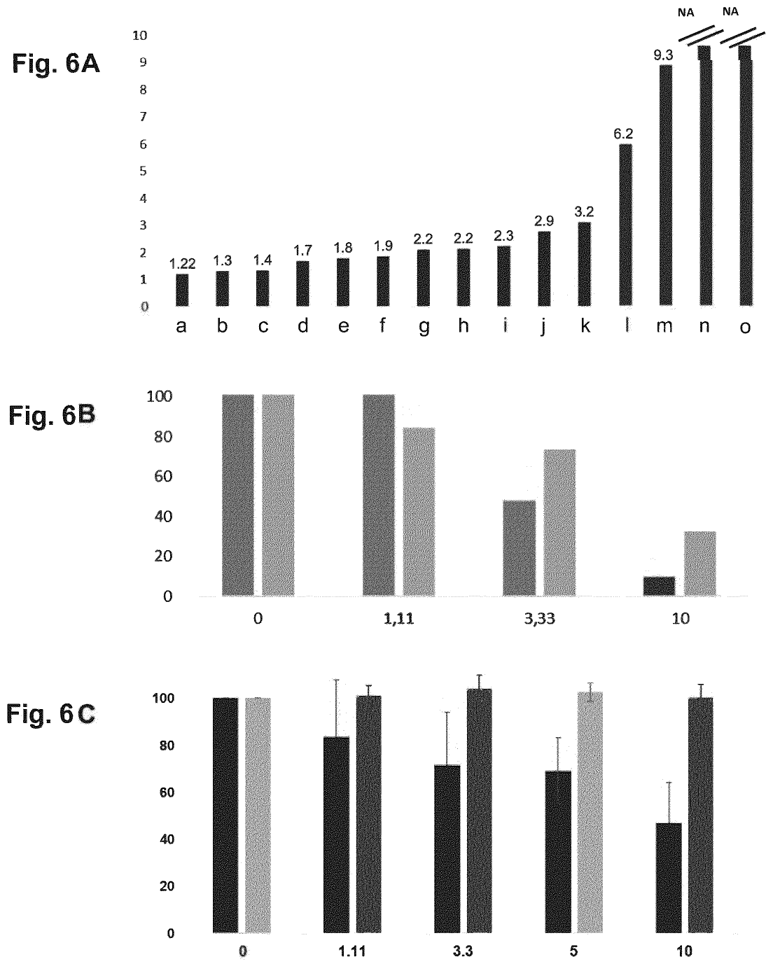

The inventors then investigated the therapeutic interest of G4 stabilizers to kill MM cells. Treatment with the G4 stabilizer Pyridostatin (PDS) was found to be associated with significant toxicity in 10 HMCLs with an IC50 s 2 uM whereas 5 cell lines demonstrated higher resistance to PDS (FIG. 6A). PDS inhibits MM cell growth and induces apoptosis with significant accumulation in G2/M phase of the cell cycle. Importantly, experiments confirmed the toxicity of PDS on primary MM cells of patients cocultured with their bone marrow microenvironment (FIG. 6B). Interestingly, the inventors confirmed that toxicity of PDS on primary malignant PCs of MM patients, without any significant toxicity on the co-cultured normal cells from the bone marrow microenvironment (FIG. 6C).

### Rnase H Expression in MM Cell Line

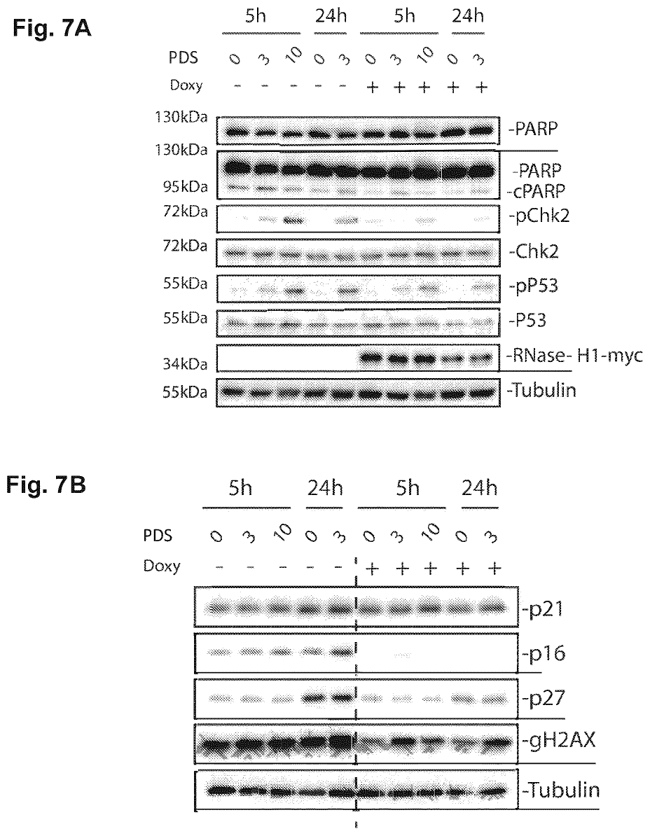

Inducible RNase H expression in MM cell line resulted in a significant reduction of DNA damage response after PDS treatment with a decreased phosphorylation of Chk2, p53 and a decrease of p21 and p16 expression (FIG. 7). Since RNase H specifically degrades RNA:DNA hybrids, this result supports the view that spontaneous replication stress and genomic instability are caused by R-loops in MM cells.

### Bet Proteins Inhibitors' Sensitivity in MM Cell Lines

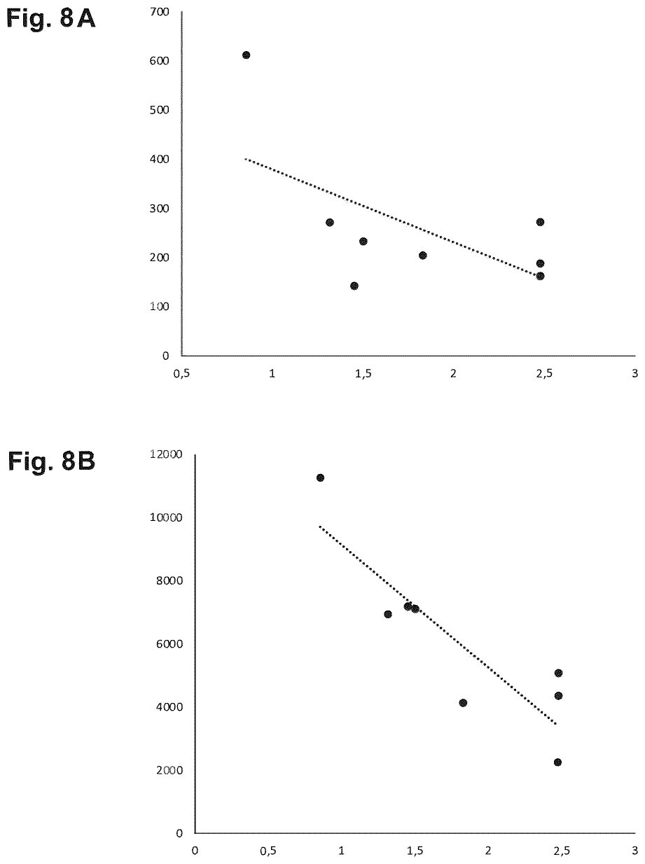

The inventors also found a correlation between HMCLs TRC score and the response to two Bromodomain and Extra-Terminal motif (BET) proteins inhibitors, I-BET-762 and RVX-208 (FIGS. 8A and 8B). Without being bound by any theory, the inventors suggest that this result could be explained by that BET proteins inhibition may increase R-loop formation and DNA damage.

Overall, these results demonstrate the ability of the score TRC_{score} to identify interesting drug combinations using PDS in MM cells.

Drugs Combination with G4 Stabilizer

The inventors investigated the sensitivity of HMCLs for different drugs combination with G4 stabilizer: HDAC inhibitors and BET proteins inhibitors.

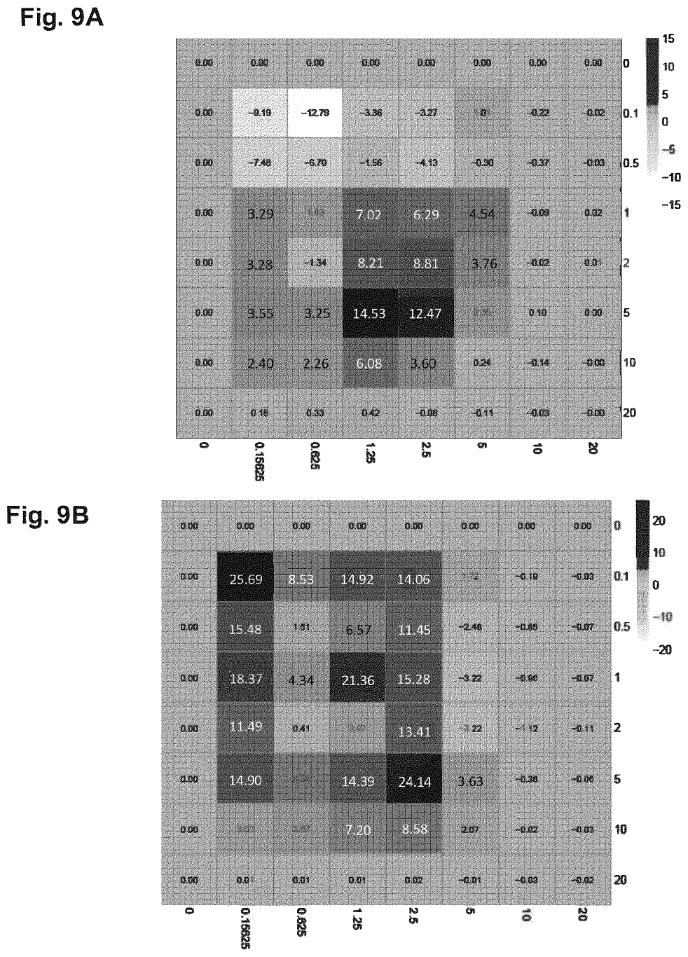

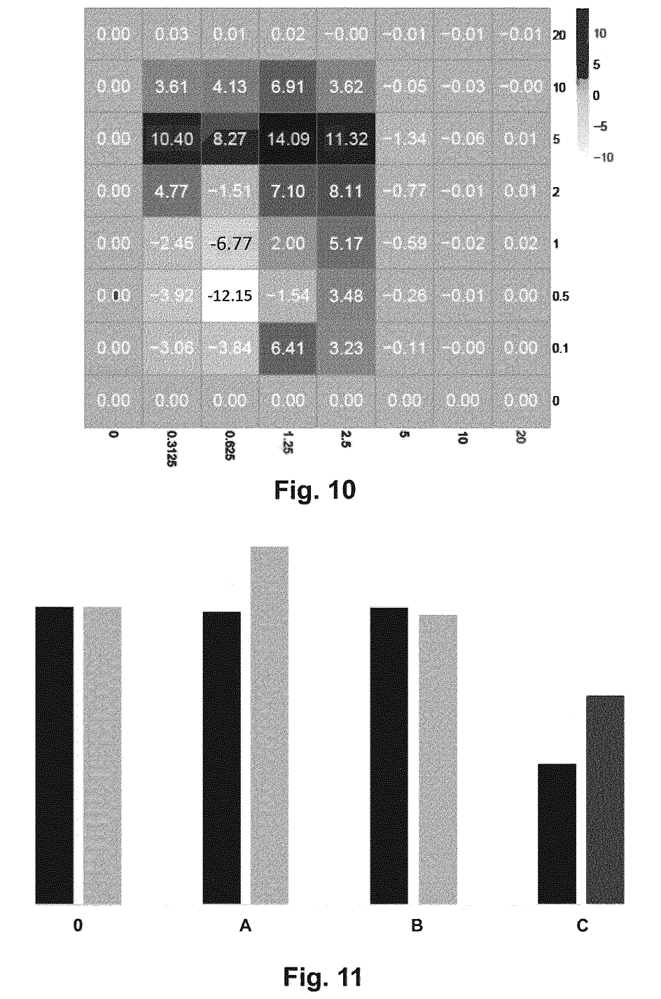

The combination of PDS and Panobinostat was found to be a synergistic effect in three HMCLs (FIGS. 9 and 10). Moreover, it was showed the absence of too much toxicity of this combination on primary samples from MM patients (FIG. 11).

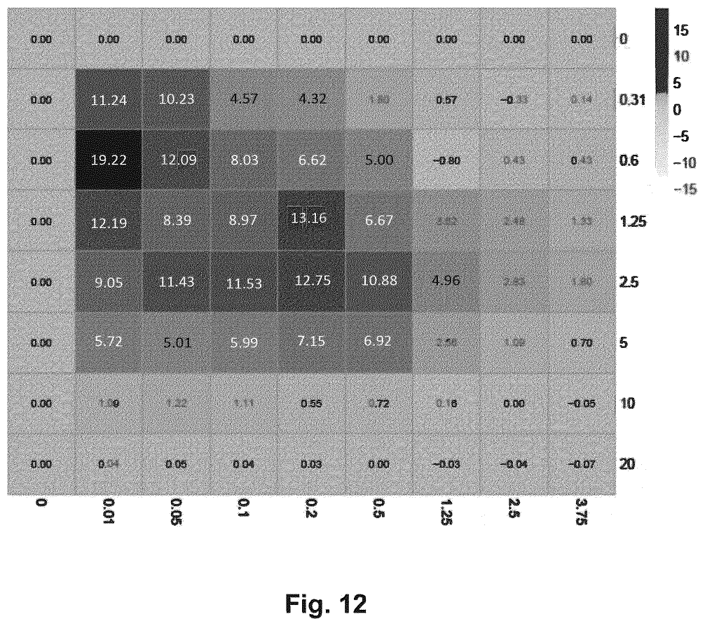

Furthermore, the synergistic effect of the combination of PDS with I-BET-762 was confirmed for one cell line (FIG. 12).

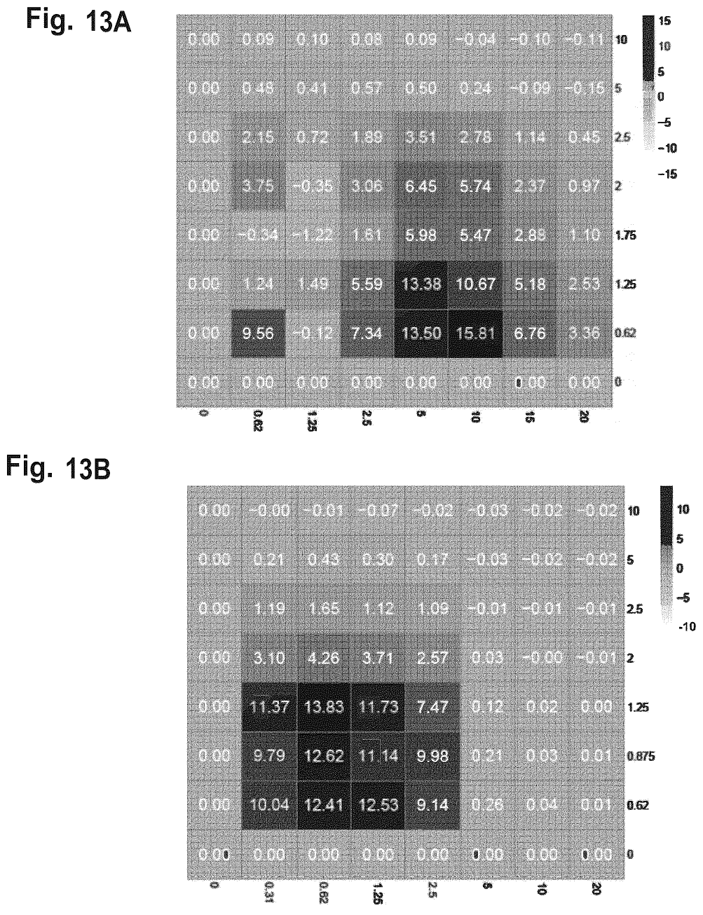

The combination of PDS and Melphalan was found to show a synergistic effect in two HMCLs (FIG. 13). These results underline that PDS can increase the DNA damage induced by Melphalan.

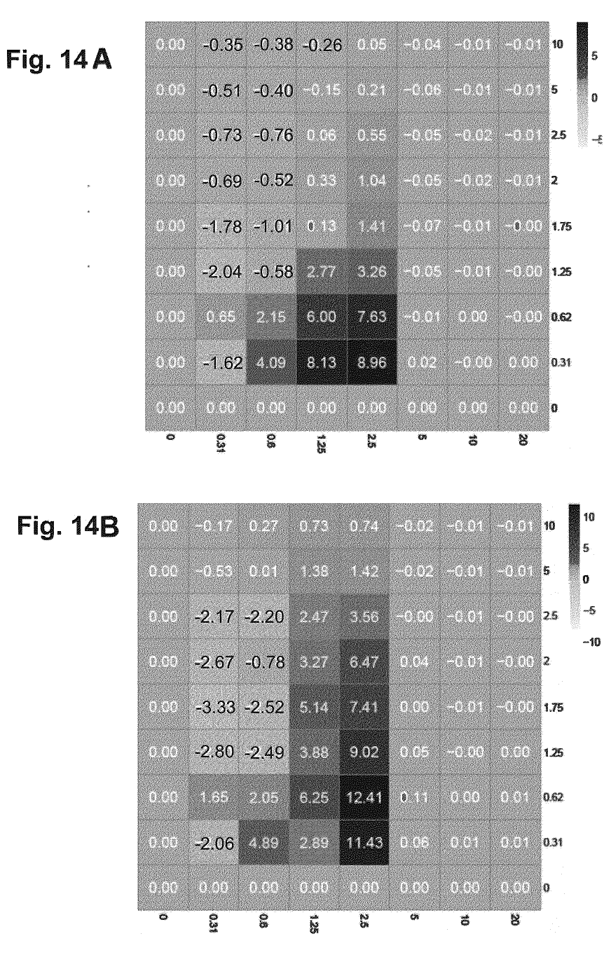

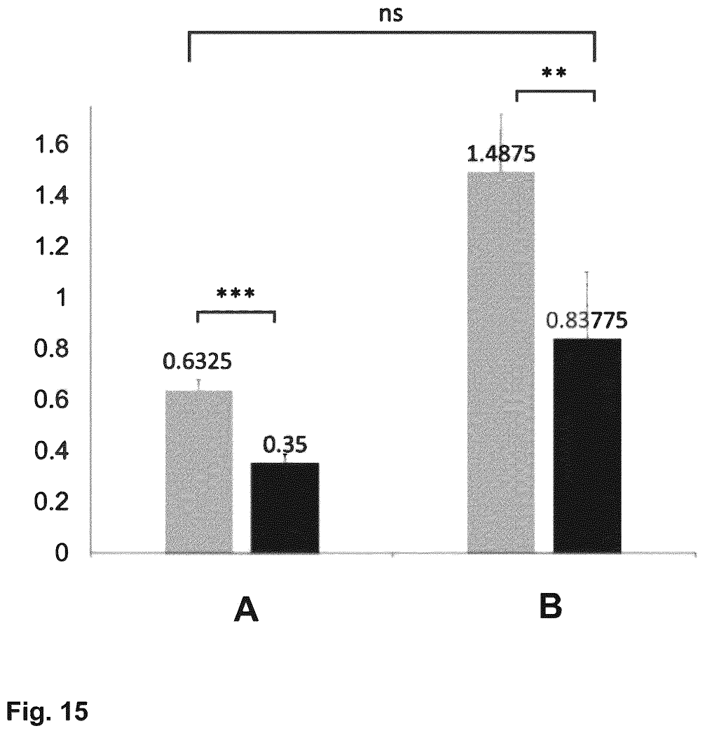

At that stage the inventors hypothesize that PDS could be used as a strategy to increase MM patients' response to Melphalan. This is demonstrated in FIGS. 14 and 15 where PDS is shown to re-sensitize the resistant cells (XG2-MeIR) to Melphalan.

### Ddx1 and Ddx23 Depletions

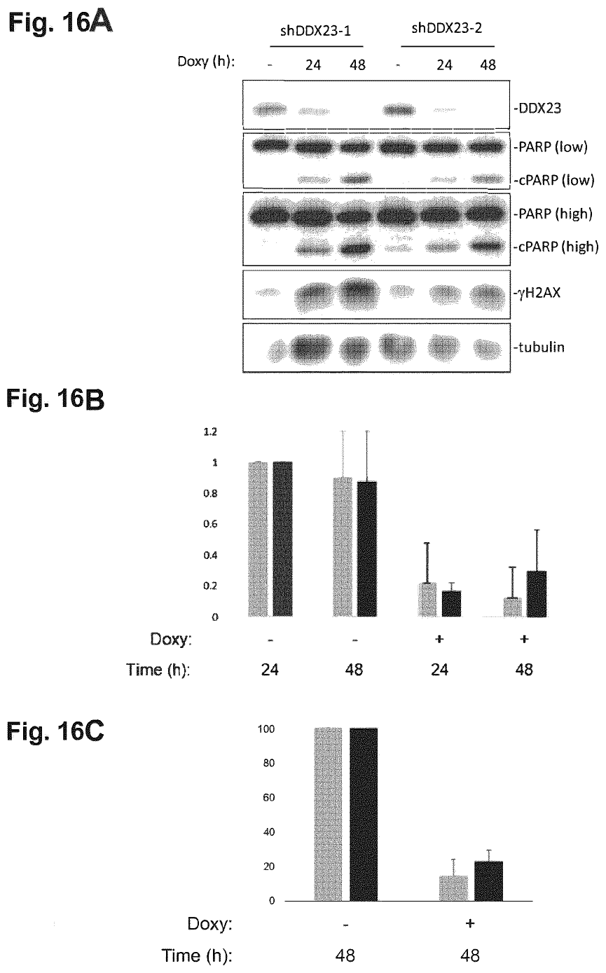

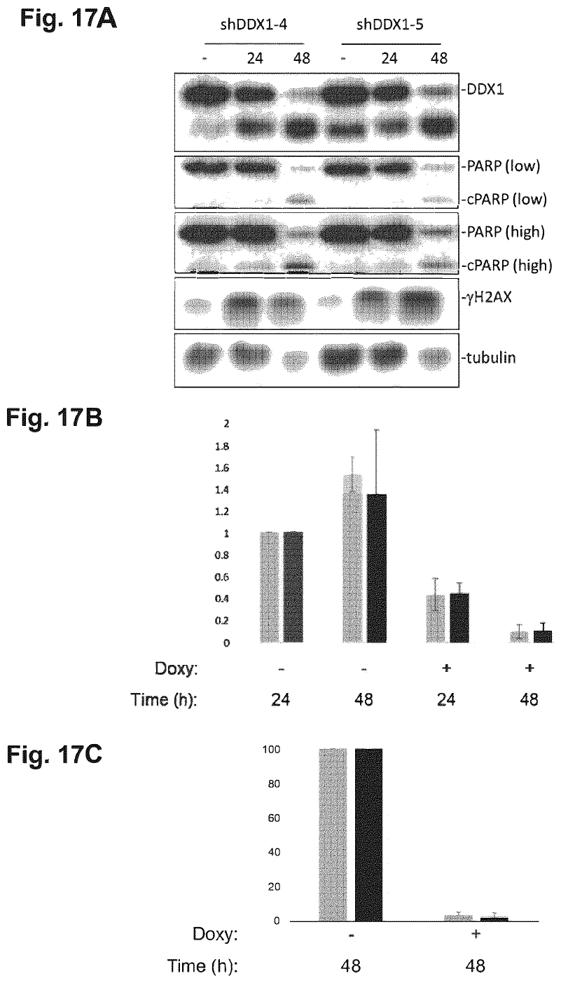

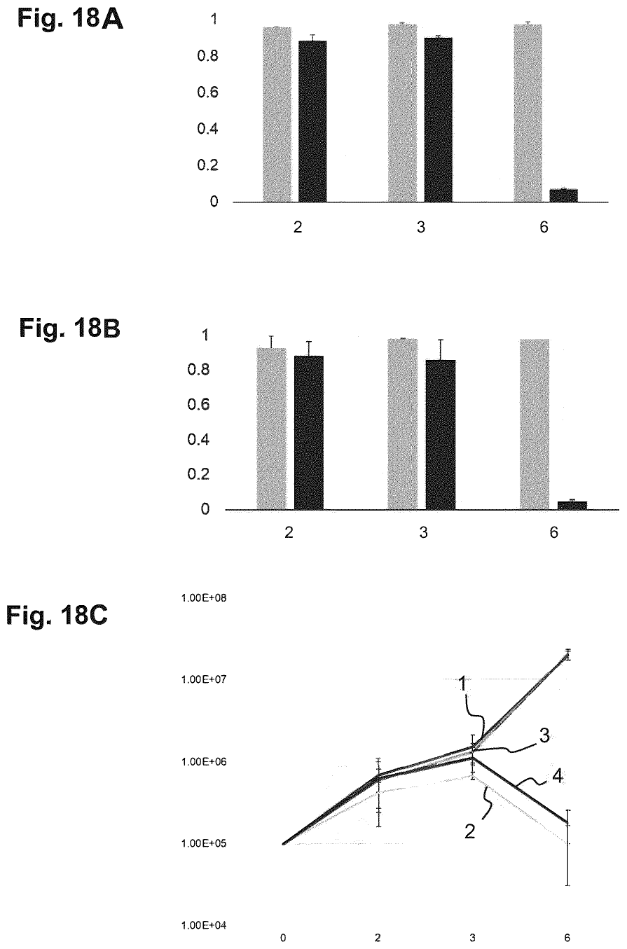

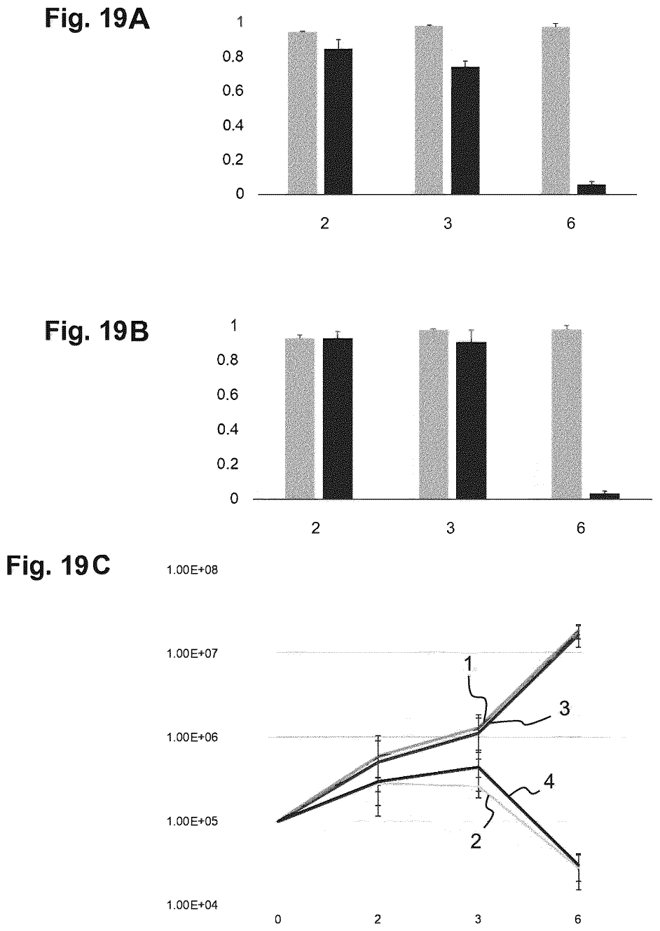

The inventors searched to validate the pathophysiological roles of two genes of the TRC score, namely DDX1 and DDX23. To that purpose, the inventors transduced XG7 cells with inducible shRNAs targeting either DDX1 or DDX23. The inventors validated the depletion of the two proteins by western-blot (FIGS. 16A, B and 17A, B) and RT-qPCR (FIGS. 16C and 17C). The inventors found that the depletion of DDX1 affected cell viability and decreased cell proliferation (FIG. 19) whereas DDX23 depletion had a mild effect on cell viability and proliferation (FIG. 18).

Moreover, the inventors found that DDX1 and DDX23 depletions led to an increased phosphorylation of γH2AX which points out spontaneous DNA damage formation (FIGS. 16A and 17A). Together, these results show that DDX1 and DDX23 are critical for MM cells survival and in TRCs prevention.

1. A composition comprising at least one G-quadruplex (G4) stabilizer for its use in a method for treating an individual afflicted by a multiple myeloma.

2. The composition according to claim 1, wherein the at least one G4 stabilizer is selected in a group consisting of Quarfloxin, Pidnarulex, MM41, Telomestatin, BMSG-SH-3, BRACO-19, CMO3, PDP, Pyridostatin, carboxyPyridostatin, PhenDC3, AQ1, TMPyP4, RHPS4, 360A, FG, 20A, Emetine and combinations thereof, preferably the G4 stabilizer is Pyridostatin.

3. The composition according to claim 1, wherein the composition further comprises at least one histone deacetylase inhibitor.

4. The composition according to claim 3, wherein the at least one histone deacetylase inhibitor is selected in a group consisting of Panobinostat, trichostatin-A, vorinostat, belinostat, NVP-LAQ824, givinostat, romidepsin, valproic acid, sodium phenylbutyrate, MS-275, MGCD0103 and combinations thereof, preferably the histone deacetylase inhibitor is Panobinostat.

5. The composition according to claim 1, wherein the composition further comprises at least one bromodomain and extraterminal (BET) proteins inhibitor.

6. The composition according to claim 5, wherein the at least one BET proteins inhibitor is selected in the group consisting of RVX-208, I-BET-762, OTX015, CPI-0610, GSK525762, ABBV-075, FT-1101, INCB057643, ZEN003694, GSK2820151, CC-90010, PLX51107, ABBV-744, BAY1238097, B1894999, BMS-986158, GS-5829, INCB054328, RO6870810 and combinations thereof.

7. The composition according to claim 1, wherein the composition further comprises at least one nitrogen mustard.

8. The composition according to claim 7, wherein the nitrogen mustard is selected in the group consisting of Chlormethine, Chlorambucil, Melphalan, Cyclophosphamide, Ifosfamide, Estramustine, Prednimustine, Bendamustine, Melphalan flufenamide and combinations thereof.

9. The composition according to claim 1, wherein the individual has been prognosed with a poor outcome.

10. The composition according to claim 9, wherein the poor outcome of the individual is in vitro determined by carrying out the following steps:
a) measuring in a biological sample from said individual, the expression level of 9 genes consisting of the nucleic acid sequences SEQ ID NO:1 to SEQ ID NO:9;
b) calculating a score TRC_{score} according to the following formula
$$T R {C}_{s c o r e} = {\sum }_{i = 1}^{9} β i \times C i$$
wherein βi represents the regression β coefficient reference value for the gene of nucleic acid sequence SEQ ID NO:i,
wherein Ci=1 if the expression level of the gene of nucleic acid sequence SEQ ID NO:i is higher than an expression level of reference Ci=−1 if the expression level of the gene of nucleic acid sequence SEQ ID NO:i is lower than or equal to ELRi,
c) prognosing that
said individual with a score TCR_{score} higher than a reference value TRC_{ref} is likely to have a bad outcome, or
said individual with a score TCR_{score} lower than a reference value TRC_{ref} is likely to have a good outcome,
wherein the reference value TRC_{ref} is of −0.39535.


SEQUENCE LISTING (9 sequences)

SEQ ID NO: 1 - DNA, 3750 bp, Homo sapiens
  source 1..3750: mol_type = genomic DNA
atgtcttcaatgtggtctgaatatacaattggtggggtgaagatttactttccttataaagcttacccgtcacagcttgctatgatgaattctattctcagaggattaaacagcaagcaacattgtttgttggagagtcccacaggaagtggaaaaagcttagccttactttgttctgctttagcatggcaacaatctcttagtgggaaaccagcagatgagggcgtaagtgaaaaagctgaagtacaattgtcatgttgttgtgcatgccattcaaaggattttacaaacaatgacatgaaccaaggaacttcacgtcatttcaactatccaagcacaccaccttctgaaagaaatggcacttcatcaacttgtcaagactcccctgaaaaaaccactctggctgcaaagttatctgctaagaaacaggcatccatatacagagatgaaaatgatgattttcaagtagagaagaaaagaattcgacccttagaaactacacagcagattagaaaacgtcattgctttggaacagaagtacacaatttggatgcaaaagttgattcaggaaagactgtaaaactcaactctccactggaaaagataaactccttttcgccacagaaaccccctggccactgttctaggtgctgttgttctactaaacaaggaaacagtcaagagtcatcgaataccattaagaaggatcatacagggaaatccaagatacccaaaatatattttgggacacgcacacacaagcagattgctcagattactagagagctccggaggacggcatattcaggggttccaatgactattctttccagcagggatcatacttgtgtccatcctgaggtagtcggtaacttcaacagaaatgagaagtgcatggaattgctagatgggaaaaacggaaaatcctgctatttttatcatggagttcataaaattagtgatcagcacacattacagactttccaagggatgtgcaaagcctgggatatagaagaacttgtcagcctggggaagaaactaaaggcctgtccatattacacagcccgagaactaatacaagatgctgacatcatattttgtccctacaactatcttctagatgcacaaataagggaaagtatggatttaaatctgaaagaacaggttgtcattttagatgaagctcataacatcgaggactgtgctcgggaatcagcaagttacagtgtaacagaagttcagcttcggtttgctcgggatgaactagatagtatggtcaacaataatataaggaagaaagatcatgaacccctacgagctgtgtgctgtagcctcattaattggttagaagcaaacgctgaatatcttgtagaaagagattatgaatcagcttgtaaaatatggagtggaaatgaaatgctcttaactttacacaaaatgggtatcaccactgctacttttcccattttgcagggacatttttctgctgttcttcaaaaagaggaaaaaatctcaccaatttatggtaaagaggaggcaagagaagtacctgttattagtgcatcaactcaaataatgcttaaaggactttttatggtacttgactatctttttaggcaaaatagcagatttgcagatgattataaaattgcgattcaacagacttactcctggacaaatcagattgatatttcagacaaaaatgggttgttggttctaccaaaaaataagaaacgttcacgacagaaaactgcagttcatgtgctaaacttttggtgcttaaatccagctgtggccttttcagatattaatggcaaagttcagaccattgttttgacatctggtacattatcaccaatgaaatccttttcgtcagaacttggtgttacatttactatccagctggaggctaatcatatcattaaaaattcacaggtttgggttggtaccattgggtcaggccccaagggtcggaatctctgtgctaccttccagaatactgaaacatttgagttccaagatgaagtgggagcacttttgttatctgtgtgccagactgtgagccaaggaattttgtgtttcttgccatcttacaagttattagaaaaattaaaagaacgttggctctctactggtttatggcataatctggagttggtgaagacagtcattgtagaaccacagggaggagaaaaaacaaattttgatgaattactgcaggtgtactatgacgcaatcaaatacaaaggagagaaagatggagctctcctggtagcagtttgtcgtggtaaagtgagtgagggtctggatttctcagatgacaatgcccgtgctgtcataacaataggaattccttttccaaatgtgaaagatctacaggttgaactaaaacgacaatacaatgaccaccattcaaaattgagaggtcttctacctggccgtcagtggtatgaaattcaagcatacagggccttaaaccaggcccttggtagatgtattagacacagaaatgattggggagctcttattctagtggatgatcgctttaggaataacccaagtcgctatatatctggactttctaaatgggtacggcagcagattcagcaccattcaacctttgaaagtgcactggaatccttggctgaattttccaaaaagcatcaaaaagttcttaatgtatccataaaggacagaaccaatatacaggacaatgagtctacacttgaagtgacctctttaaagtacagtacctcaccttatttactggaagcagcaagtcatctatcaccagaaaattttgtggaagatgaagcaaagatatgtgtccaggaactacagtgtcctaaaattattaccaaaaattcacctctaccaagtagcattatctccagaaaggagaaaaatgatccagtattcctggaagaagcagggaaagcagaaaaaattgtgatttccagatccacaagcccaactttcaacaaacaaacaaagagagttagctggtcaagctttaattctttgggacagtattttactggtaaaataccgaaggcaacacctgagctcgggtcatcagagaatagtgcctctagtcctccccgtttcaaaacagagaagatggaaagtaaaactgttttgcccttcactgataaatgtgaatcctcaaatctgacagtaaacacatcgtttggatcatgccctcaatcagaaaccattatttcatcattaaagattgatgccacccttactagaaaaaatcattctgaacatccgctctgttctgaagaagccctggatccagacattgaattgtctctagtaagtgaagaagataaacagtccacttcaaatagagattttgaaacagaagcagaagatgaatctatctattttacacctgaactttatgatcctgaagatacagatgaagaaaaaaatgacctagctgaaactgatagaggaaatagattggctaacaattcagattgcattttagctaaagacctttttgaaattagaactataaaagaagtagattcagccagagaagtgaaagctgaggattgcatagatacaaagttgaatggaattctgcatattgaagaaagtaaaattgatgacattgatggtaatgtaaaaacaacttggataaatgaactggaactgggaaaaactcatgaaatagaaataaagaactttaaaccatctccttccaaaaataaaggcatgtttcctggttttaagtaa

SEQ ID NO: 2 - DNA, 2223 bp, Homo sapiens
  source 1..2223: mol_type = genomic DNA
atggcggccttctccgagatgggtgtaatgcctgagattgcacaagctgtggaagagatggattggctcctcccaactgatatccaggctgaatctatcccattgatcttaggaggaggtgatgtacttatggctgcagaaacaggaagtggcaaaactggtgcttttagtattccagttatccagatagtttatgaaactctgaaagaccaacaggaaggcaaaaaaggaaaaacaacaattaaaactggtgcttcagtgctgaacaaatggcagatgaacccatatgacagaggatctgcttttgcaattgggtcagatggtctttgttgtcaaagcagagaagtaaaggaatggcatgggtgtagagctactaaaggattaatgaaagggaaacactactatgaagtatcctgtcatgaccaagggttatgcagggtcgggtggtctaccatgcaggcctctttggacctaggtactgacaagtttggatttggctttggtggaacaggaaagaaatcccataacaaacaatttgataattatggagaggaattcactatgcatgataccattggatgttacctggatatagataagggacatgtcaagttctccaaaaatggaaaagatcttggtctggcatttgaaataccaccacatatgaaaaaccaagccctctttcctgcctgtgttttgaagaatgctgaactgaaatttaacttcggtgaagaggaatttaagtttccaccaaaagatggctttgttgctctttccaaggcaccggatggttacattgtcaaatcacagcactcaggtaatgcacaggtgacacaaacaaagtttctccccaatgctccgaaagctctcattgttgaaccttcccgggagttagctgaacaaactttgaacaacatcaagcagtttaagaaatacattgataatcctaaattaagggagcttctgataattggaggtgttgcagcccgggatcagctctctgttttggaaaatggagtagatatagttgtaggtactccgggaagactagatgacttggtgtcaactggaaagctgaacttatctcaagttagattcctggtcctggatgaagctgatgggcttctttctcaaggttattctgattttataaataggatgcacaatcagattcctcaggttacctctgatggaaaaagacttcaggtgattgtttgctctgccactttgcattctttcgatgtaaagaaactgtccgagaagataatgcattttcctacatgggttgacttaaaaggagaagactctgttccagatactgtacaccatgttgttgtcccagtaaatcccaaaactgacagactctgggaaaggcttggaaagagccacattagaactgatgatgtacatgcaaaagataacacaagacctggtgctaatagtccagagatgtggtctgaagctattaaaatcctgaaaggggagtatgctgtccgggcaatcaaggaacataagatggatcaagcaattatcttctgtagaaccaaaattgactgtgataacttggagcagtactttatacaacaaggaggaggacctgataaaaaaggacaccagttctcatgtgtttgtcttcatggtgacagaaagcctcatgagagaaagcaaaacttggaaagatttaagaaaggagatgtaagattcttgatttgcacagatgtagctgctagaggaattgatatccacggtgttccttatgttataaatgtcactctgcccgatgaaaagcaaaactacgtacatcgaattggcagagtaggaagagctgaaaggatgggtctggcaatttccctggtggcaacagaaaaagaaaaggtttggtaccatgtatgtagcagccgtggaaaagggtgttataacacaagactcaaggaagatggaggctgtaccatatggtacaacgagatgcagttactatctgagatagaagaacacctgaactgtaccatttctcaggttgagccggatataaaggtaccagtggatgaatttgatgggaaagttacctacggtcagaaaagggctgctggtggtggaagctataaaggccatgtggatattttggcacctactgttcaagagttggctgcccttgaaaaggaggcgcagacatctttcctgcatcttggctaccttcctaaccagctgttcagaaccttctga

SEQ ID NO: 3 - DNA, 2463 bp, Homo sapiens
  source 1..2463: mol_type = genomic DNA
atggcaggagagctggctgacaaaaaggaccgtgatgcatcaccttccaaggaggaaaggaagcgatcacggactcctgacagagagcgggatagagaccgggaccggaagtcttccccatctaaagatagaaagcggcatcgttcaagggatagacgtcgaggaggcagccgttctcgctctcgttcccgttccaaatctgcagaaagagaacgacggcacaaagaacgagaacgagataaggagcgggatcggaataagaaggaccgagatcgagacaaggatgggcacagacgggacaaggaccgtaaacgatccagcttatctcctggtcgaggaaaagactttaaatctcggaaggacagagactctaagaaggatgaagaggatgaacatggtgataagaagcctaaggcccagccattatccctggaggagcttctggccaagaaaaaggctgaggaagaagctgaggctaagcccaagttcctctctaaagcagaacgagaggctgaagctctaaagcgacggcagcaggaggtggaagagcggcagaggatgcttgaagaagagaggaagaaaaggaaacagttccaagacttgggcaggaagatgttggaagatcctcaggaacgggaacgtcgggaacgcagggagaggatggaacgggagaccaatggaaatgaggatgaggaagggcggcagaagatccgggaagagaaggataagagcaaggaactgcatgccattaaggagcgttacctgggtggcatcaaaaagcggcgccgaacgagacatctcaatgaccggaaatttgtttttgagtgggatgcatctgaggacacatccattgactacaaccccctgtacaaagaacggcaccaggtgcagttgttagggcgaggcttcattgcaggcattgacctcaagcagcagaagcgagagcagtcacgtttctatggagacctaatggagaagaggcgaaccctggaagaaaaggagcaggaggaggcaagactccgcaaacttcgtaagaaggaagccaagcagcgctgggatgatcgtcattggtctcagaaaaagttagatgagatgacggacagggactggcggatcttccgtgaggactacagcatcaccaccaaaggtggcaagatccccaatcccatccgatcctggaaagactcttctctgcccccacacatcttggaggtcattgataagtgtggctacaaggaaccaacacctatacagcgtcaggcaattcccattgggctacagaatcgtgacatcattggtgtggctgagactggcagtggcaagacagcagccttcctcatccctctgctggtctggatcaccacacttcccaaaattgacaggatcgaagagtcagaccaaggcccttatgccatcatcctggctcccacccgtgagttggctcaacagattgaggaagagaccatcaagtttgggaaaccgctaggtatccgcactgtggctgtcattggtggcatctccagagaagaccagggcttcaggctgcgcatgggttgtgagattgtgattgctacccctgggcgtttgattgatgtgctggagaaccgctacctggtgctgagccgctgtacctatgtggttctggatgaggcagataggatgattgacatgggctttgagccagatgtccagaagatcctggagcacatgcctgtcagcaaccagaagccagacacggatgaggctgaggaccctgagaagatgctggccaactttgagtcgggaaaacataagtaccgccaaacagtcatgttcacggccaccatgcccccagcggtggagcgtctggccaggagctatcttcggcgacctgctgtggtgtacattggctccgcaggcaagccccatgagcgtgtggaacagaaggtcttcctcatgtcagagtcagaaaagaggaaaaagctgctggcaatcttggagcaaggctttgacccacccatcattatttttgtcaaccagaagaagggctgcgacgtgttggccaaatccctggagaagatggggtacaatgcttgcacactgcacggtggaaaaggccaggagcagcgagagtttgcgttgtccaacctcaaggctggggccaaggatattttggtggctacagatgtggctggtcgtggtattgacatccaagatgtgtctatggttgtcaactatgatatggccaaaaatattgaagattacatccaccgcattggccgcacgggacgagcaggcaagagtggggtggccatcaccttcctcacaaaagaggactctgctgtgttctacgagctgaagcaagctatcctggaaagcccagtgtcttcctgtccccccgaactagccaaccacccagatgcccagcataagccaggcaccatcctcaccaagaagcgccgggaagagaccatctttgcctga

SEQ ID NO: 4 - DNA, 708 bp, Homo sapiens
  source 1..708: mol_type = genomic DNA
atggaggaggagacgcatactgacgccaaaatccgtgctgaaaatggaacagggtccagccctcggggtcctggctgcagcctccggcactttgcctgcgaacagaacctgctgtcgcggccagatggctctgcttccttcctgcaaggtgacacctctgtcctggcgggtgtgtacgggccggccgaggtgaaggtcagcaaagagattttcaacaaggccacactcgaagtgatcctgaggccgaagattgggctgcctggtgttgcagagaagagccgggagcggctgatcaggaacacgtgcgaggcggtggtgctgggcacgttgcacccccgcacctccatcaccgtggtgctgcaggttgtcagcgatgccggctctctcctggcctgttgtctgaatgccgcctgcatggcattggtggatgcaggtgtgcccatgcgggctctcttctgtggggtcgcctgcgccctggactctgatgggaccctcgtgctggatcctacatccaagcaagaaaaggaggcccgggcagtcctgacctttgccctggacagcgtggaacggaagctgctgatgtccagcaccaaggggctctactcagacactgagctccagcagtgcctggctgcggcccaggccgcttcgcaacacgtcttccgtttctaccgggaatcgctgcagaggcgttactccaagagctga

SEQ ID NO: 5 - DNA, 4416 bp, Homo sapiens
  source 1..4416: mol_type = genomic DNA
atggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggtatctctacaaaacccaccagagtctggcactgatggttgcattttgttaattgttctaagttggtggagcagaactttgcctacttatgtttattgtcaaatgcttctatgcccatttccattccctccataa

SEQ ID NO: 6 - DNA, 2478 bp, Homo sapiens
  source 1..2478: mol_type = genomic DNA
atgagttcctcgcctgttaatgtaaaaaagctgaaggtgtcggagctgaaagaggagctcaagaagcgacgcctttctgacaagggtctcaaggccgagctcatggagcgactccaggctgcgctggacgacgaggaggccgggggccgccccgccatggagcccgggaacggcagcctagacctgggcggggattccgctgggcgctcgggagcaggcctcgagcaggaggccgcggccggcggcgatgaagaggaggaggaagaggaagaggaggaggaaggaatctccgctctggacggcgaccagatggagctaggagaggagaacggggccgcgggggcggccgactcgggcccgatggaggaggaggaggccgcctcggaagacgagaacggcgacgatcagggtttccaggaaggggaagatgagctcggggacgaagaggaaggcgcgggcgacgagaacgggcacggggagcagcagcctcaaccgccggcgacgcagcagcaacagccccaacagcagcgcggggccgccaaggaggccgcggggaagagcagcggccccacctcgctgttcgcggtgacggtggcgccgcccggggcgaggcagggccagcagcaggcgggaggtaagaagaaggcggaaggcggcggaggcggcggtcgccccggggctccggcggcgggggacggcaaaacagaacagaaaggcggagataaaaagaggggtgttaaaagaccacgagaagatcatggccgtggatattttgagtacattgaagagaacaagtatagcagagccaaatctcctcagccacctgttgaagaagaagatgaacacttcgatgacacagtggtttgtcttgatacttataattgtgatctacattttaaaatatcaagagatcgtctcagtgcttcttcccttacaatggagagttttgcttttctttgggctggaggaagagcatcctatggtgtgtcaaaaggcaaagtgtgttttgagatgaaggttacagagaagatcccagtaaggcatttatatacaaaagatattgacatacatgaagttcgtattggctggtcactaactacaagtggaatgttacttggtgaagaagaattttcttatgggtattctctaaaaggaataaaaacatgcaactgtgagactgaagattatggagaaaagtttgatgaaaatgatgtgattacatgttttgctaactttgaaagtgatgaagtagaactctcgtatgctaagaatggacaagatcttggcgttgccttcaaaatcagtaaggaagttcttgctggacggccactgttcccgcatgttctctgccacaactgtgcagttgaatttaattttggtcagaaggaaaagccatattttccaatacctgaagagtatactttcatccagaacgtccccttagaggatcgagttagaggaccaaaggggcctgaagagaagaaagattgtgaagttgtgatgatgattggcttgccaggagctggaaaaactacctgggttactaaacatgcagcagaaaatccagggaaatataacattcttggcacaaatactattatggataagatgatggtggcaggttttaagaagcaaatggcagatactggaaaactgaacacactgttgcagagagccccccagtgtcttgggaaatttattgagattgctgcccgaaagaagcgaaattttattctggatcagacaaatgtgtctgctgctgcccagaggagaaaaatgtgcctgtttgcaggcttccagcgaaaagctgttgtagtttgcccaaaagatgaagactataagcaaagaacacagaagaaagcagaagtagaggggaaagacctaccagaacatgcggtcctcaaaatgaaaggaaactttaccctcccagaggtagctgagtgctttgatgaaataacctatgttgaacttcagaaggaagaagcccaaaaactcttggagcaatataaggaagaaagcaaaaaggctcttccaccagaaaagaaacagaacactggctcaaagaaaagcaataaaaataagagtggcaagaaccagtttaacagaggtggtggccatagaggacgtggaggattcaatatgcgtggtggaaatttcagaggaggagcccctgggaatcgtggcggatataataggaggggcaacatgccacagagaggtggtggcggtggaggaagtggtggaatcggctatccataccctcgtgcccctgtttttcctggccgtggtagttactcaaacagagggaactacaacagaggtggaatgcccaacagagggaactacaaccagaacttcagaggacgaggaaacaatcgtggctacaaaaatcaatctcagggctacaaccagtggcagcagggtcaattctggggtcagaagccatggagtcagcattatcaccaaggatattattga

SEQ ID NO: 7 - DNA, 1914 bp, Homo sapiens
  source 1..1914: mol_type = genomic DNA
atggcggcgatggcggtcgggggtgctggtgggagccgcgtgtccagcgggagggacctgaattgcgtccccgaaatagctgacacactaggggctgtggccaagcaggggtttgatttcctctgcatgcctgtcttccatccgcgtttcaagagggagttcattcaggaacctgctaagaatcggcccggtccccagacacgatcagacctactgctgtcaggaagggactggaatacgctaattgtgggaaagctttctccatggattcgtccagactcaaaagtggagaagattcgcaggaactccgaggcggccatgttacaggagctgaattttggtgcatatttgggtcttccagctttcctgctgccccttaatcaggaagataacaccaacctggccagagttttgaccaaccacatccacactggccatcactcttccatgttctggatgcgggtacccttggtggcaccagaggacctgagagatgatataattgagaatgcaccaactacacacacagaggagtacagtggggaggagaaaacgtggatgtggtggcacaacttccggactttgtgtgactatagtaagaggattgcagtggctcttgaaattggggctgacctcccatctaatcatgtcattgatcgctggcttggggagcccatcaaagcagccattctccccactagcattttcctgaccaataagaagggatttcctgttctttctaagatgcaccagaggctcatcttccggctcctcaagttggaggtgcagttcatcatcacaggcaccaaccaccactcagagaaggagttctgctcctacctccaatacctggaatacttaagccagaaccgtcctccacctaatgcctatgaactctttgccaagggctatgaagactatctgcagtccccgcttcagccactgatggacaatctggaatctcagacatatgaagtgtttgaaaaggaccccatcaaatactctcagtaccagcaggccatctataaatgtctgctagaccgagtaccagaagaggagaaggataccaatgtccaggtactgatggtgctgggagcaggacggggacccctggtgaacgcttccctgcgggcagccaagcaggccgaccggcggataaagctgtatgctgtggagaaaaacccaaatgccgtggtgacgctagagaactggcagtttgaagaatggggaagccaagtgaccgtagtctcatcagacatgagggaatgggtggctccagagaaagcagacatcattgtcagtgagcttctgggctcatttgctgacaatgaattgtcgcctgagtgcctggatggagcccagcacttcctaaaagatgatggtgtgagcatccccggggagtacacttcctttctggctcccatctcttcctccaagctgtacaatgaggtccgagcctgtagggagaaggaccgtgaccctgaggcccagtttgagatgccttatgtggtacggctgcacaacttccaccagctctctgcaccccagccctgtttcaccttcagccatcccaacagagatcctatgattgacaacaaccgctattgcaccttggaatttcctgtggaggtgaacacagtactacatggctttgccggctactttgagactgtgctttatcaggacatcactctgagtatccgtccagagactcactctcctgggatgttctcatggtttcccatcctcttccctattaagcagcccataacggtacgtgaaggccaaaccatctgtgtgcgtttctggcgatgcagcaattccaagaaggtgtggtatgagtgggctgtgacagcaccagtctgttctgctattcataaccccacaggccgctcatataccattggcctctag

SEQ ID NO: 8 - DNA, 2100 bp, Homo sapiens
  source 1..2100: mol_type = genomic DNA
atgagctcccggaaagtgctggccattcaggcccgaaagcggaggccgaaaagagagaaacatccgaaaaagccggagcctcaacagaaagctcctttagttcctcctcctccaccgccaccaccaccaccaccgccacctttgccagaccccacacccccggagccagaggaggagatcctgggatcagatgatgaggagcaagaggaccctgcggactactgcaaaggtggatatcatccagtgaaaattggagacctcttcaatggccggtatcatgttattagaaagcttggatgggggcacttctctactgtctggctgtgctgggatatgcaggggaaaagatttgttgcaatgaaagttgtaaaaagtgcccagcattatacggagacagccttggatgaaataaaattgctcaaatgtgttcgagaaagtgatcccagtgacccaaacaaagacatggtggtccagctcattgacgacttcaagatttcaggcatgaatgggatacatgtctgcatggtcttcgaagtacttggccaccatctcctcaagtggatcatcaaatccaactatcaaggcctcccagtacgttgtgtgaagagtatcattcgacaggtccttcaagggttagattacttacacagtaagtgcaagatcattcatactgacataaagccggaaaatatcttgatgtgtgtggatgatgcatatgtgagaagaatggcagctgaggccactgagtggcagaaagcaggtgctcctcctccttcagggtctgcagtgagtacggctccacagcagaaacctataggaaaaatatctaaaaacaaaaagaaaaaactgaaaaagaaacagaagaggcaggctgagttattggagaagcgcctgcaggagatagaagaattggagcgagaagctgaaaggaaaataatagaagaaaacatcacctcagctgcaccttccaatgaccaggatggcgaatactgcccagaggtgaaactaaaaacaacaggattagaggaggcggctgaggcagagactgcaaaggacaatggtgaagctgaggaccaggaagagaaagaagatgctgagaaagaaaacattgaaaaagatgaagatgatgtagatcaggaacttgcgaacatagaccctacgtggatagaatcacctaaaaccaatggccatattgagaatggcccattctcactggagcagcaactggacgatgaagatgatgatgaagaagactgcccaaatcctgaggaatataatcttgatgagccaaatgcagaaagtgattacacatatagcagctcctatgaacaattcaatggtgaattgccaaatggacgacataaaattcccgagtcacagttcccagagttttccacctcgttgttctctggatccttagaacctgtggcctgcggctctgtgctttctgagggatcaccacttactgagcaagaggagagcagtccatcccatgacagaagcagaacggtttcagcctccagtactggggatttgccaaaagcaaaaacccgggcagctgacttgttggtgaatcccctggatccgcggaatgcagataaaattagagtaaaaattgctgacctgggaaatgcttgttgggtgcataaacacttcacggaagacatccagacgcgtcagtaccgctccatagaggttttaataggagcggggtacagcacccctgcggacatctggagcacggcgtgtatggcatttgagctggcaacgggagattatttgtttgaaccacattctggggaagactattccagagacgaagaccacatagcccacatcatagagctgctaggcagtattccaaggcactttgctctatctggaaaatattctcgggaattcttcaatcgcagaggagaactgcgacacatcaccaagctgaagccctggagcctctttgatgtacttgtggaaaagtatggctggccccatgaagatgctgcacagtttacagatttcctgatcccgatgttagaaatggttccagaaaaacgagcctcagctggcgaatgccttcggcatccttggttgaattcttag

SEQ ID NO: 9 - DNA, 3087 bp, Homo sapiens
  source 1..3087: mol_type = genomic DNA
atgggctccgccgcctgcccccggggagccttgccggagctcgcgccctgctgccagcctcgcgagcagtcgcagccccacacgcgatgggacgcgggctgtgggattcagcaccccgggggcgaggaattcaggaccctcggcggggcaagggcctatagggttccgaactcgcaggagggtcgctcctcccctactcgctttttcccggcaccggaaggccccgcccactgctttgtttcctctccagaccgcgcattttgggtctcggaagaggttcagaggctgttgttgagcaatgcatgccagccaaaagaatgcaatggtgtaaagattccagttgatgccagtaaacctaatccaaatgatgtggagtttgataatctgtatttggatatgaatggaatcatccatccctgtactcatcctgaagacaaaccagcaccaaaaaatgaagatgaaatgatggttgcaatttttgagtacattgacagacttttcagtattgtaagaccaagaagacttctctacatggcaatagatggagtggcaccacgtgctaaaatgaaccagcagcgttcaaggaggttcagggcatcaaaagaaggaatggaagcagcagtcgagaagcagcgagtcagggaagaaatattggcaaaaggtggctttcttcctccagaagaaataaaagaaagatttgacagcaactgtattacaccaggaactgaattcatggacaatcttgctaaatgccttcgctattacatagctgatcgtttaaataatgaccctgggtggaaaaatttgacagttattttatctgatgctagtgctcctggtgaaggagaacataaaatcatggattacattagaaggcaaagagcccagcctaaccatgacccaaatactcatcattgtttatgtggagcagatgctgatctcattatgcttggccttgccacacatgaaccgaactttaccattattagagaagaattcaaaccaaacaagcccaaaccatgtggtctttgtaatcagtttggacatgaggtcaaagattgtgaaggtttgccaagagaaaagaagggaaagcatgatgaacttgccgatagtcttccttgtgcagaaggagagtttatcttccttcggcttaatgttcttcgtgagtatttggaaagagaactcacaatggccagcctaccattcacatttgatgttgagaggagcattgatgactgggttttcatgtgcttctttgtgggaaatgacttcctccctcatttgccatcgttagagattagggaaaatgcaattgaccgtttggttaacatatacaaaaatgtggtacacaaaactgggggttaccttacagaaagtggttatgtcaatctgcaaagagtacagatgatcatgttagcagttggtgaagttgaggatagcatttttaaaaagagaaaggatgatgaggacagttttagaagacgacagaaagaaaaaagaaagagaatgaagagagatcaaccagctttcactcctagtggaatattaactcctcatgccttgggttcaagaaattcaccaggttctcaagtagccagtaatccgagacaagcagcctatgaaatgaggatgcagaataactctagtccttcgatatctcctaatacgagtttcacatctgatggctccccgtctccattaggaggaattaagcgaaaagcagaagacagtgacagtgaacctgagccagaggataatgtcaggttatgggaagctggctggaagcagcggtactacaagaacaaatttgatgtggatgcagctgatgagaaattccgtcggaaagttgtgcagtcgtacgttgaaggactttgctgggttcttagatattattaccagggctgtgcttcctggaagtggtattatccatttcattatgcaccatttgcttcagactttgaaggcattgcagacatgccatctgattttgagaagggtacgaaaccgtttaaaccactagaacaacttatgggggtatttccagctgcaagtggtaattttctacctccatcatggcggaagctcatgagtgatcctgattctagtataattgacttctatcctgaagattttgctattgatttgaatgggaagaaatatgcatggcaaggtgttgctctcttgccattcgtggatgagcgaaggctacgagctgccctagaagaggtatacccagacctcactccagaagagaccagaagaaacagccttggaggtgatgtcttatttgtggggaaacatcacccactccatgacttcattttagagctgtaccagacaggttccacagagccagtggaggtaccccctgaactatgtcatgggattcaaggaaagttttctttggatgaagaagccattcttccagatcaaatagtatgttctcctgttcctatgttaagggatctgacacagaacactgtagtcagtattaattttaaagacccacagtttgctgaagattacatttttaaagctgtaatgcttccaggagcaagaaagccagcagcagtactgaaacctagtgactgggaaaaatccagcaatggacggcagtggaagcctcagcttggctttaaccgtgaccggaggcctgtgcacctggatcaggcagccttcaggactttgggccatgtgatgccaagaggctcaggaactggcatttacagcaatgctgcaccaccacctgtgacttaccagggaaacttatacaggccgcttttgagaggacaagcccagattccaaaacttatgtcaaatatgaggccccaggattcctggcgaggtcctcctccccttttccagcagcaaaggtttgacagaggcgttggggctgaacctctgctcccatggaaccggatgctgcaaacccagaatgcagccttccagccaaaccagtaccagatgctagctgggcctggtgggtatccacccagacgagatgatcgtggagggagacagggatatcccagagaaggaaggaaataccctttgccaccaccctcaggaagatacaattggaattaa